(19) United States
(12) Patent Application Publication
(10) Pub. No.: US 2022/0372000 A1
(43) Pub. Date: Nov. 24, 2022

(54) New Process for the Synthesis of 5-Fluoro-3-(Difuoromethyl)-5-Fluoro-1-Methyl-1H-Pyrazole-4-Carboxylic Acid Derivatives and the Free Acid Thereof

(71) Applicant: Fujian Yongjing Technology Co.,Ltd, Shaowu (CN)

(72) Inventors: Weifen LUO, Shaowu (CN); Wenting WU, Shaowu (CN)

(73) Assignee: Fujian Yongjing Technology Co.,Ltd, Shaowu (CN)

(21) Appl. No.: 17/565,498

(22) Filed: Dec. 30, 2021

Related U.S. Application Data
(63) Continuation of application No. PCT/CN2021/134408, filed on Nov. 30, 2021.

(30) Foreign Application Priority Data
May 5, 2021 (DE) 102021111587.6

(51) Int. Cl. C07D 231/16

(52) U.S. Cl. CPC C07D 231/16

(57) ABSTRACT

The invention provides a new process for the synthesis of 5-fluoro-3-(difluoromethyl)-5-fluoro-1-methyl-1H-pyrazole-4-carboxylic acid derivatives and the free acid thereof, involving a direct fluorination reaction with a fluorination gas comprising or consisting of elemental fluorine (F₂), in a reactor which is resistant to elemental fluorine (F₂) and hydrogen fluoride (HF), and wherein in the process as a starting material a difluoromethyl-pyrazole compound dissolved in an inert solvent is subjected to the direct fluorination reaction. Some particular examples of 5-fluoro-3-(difluoromethyl)-5-fluoro-1-methyl-1H-pyrazole-4-carboxylic acid derivatives which can be prepared according to the process of the invention are 3-(difluoromethyl)-5-fluoro-1-methyl-1H-pyrazole-4-carboxylic acid fluoride (5F-DFMPAF), also known under the alternative name 3-(difluoromethyl)-5-fluoro-1-methyl-1H-pyrazole-4-carbonyl fluoride; 3-(difluoromethyl)-5-fluoro-1-methyl-1H-pyrazole-4-carboxylic acid ethylester (5F-DFMP); and 3-(chlorodifluoromethyl)-5-fluoro-1-methyl-1H-pyrazole-4-carboxylic acid ethylester (5F-CDFMP), or the corresponding methyl esters. The 5-fluoro-3-(difluoromethyl)-5-fluoro-1-methyl-1H-pyrazole-4-carboxylic acid can be obtained from its carboxylic acid derivatives such as, e.g., mentioned before in that the acid derivative is converted to the corresponding carboxylic acid.

### BACKGROUND OF THE INVENTION

### 1. Field of the Invention

The invention relates to a new process for the synthesis of 5-fluoro-3-(difluoromethyl)-5-fluoro-1-methyl-1H-pyrazole-4-carboxylic acid derivatives, i.e., derivatives of 5-fluoro-3-(difluoromethyl)-5-fluoro-1-methyl-1H-pyrazole-4-carboxylic acid (the free acid) wherein the carboxylic acid group is derivatized, and the free acid thereof, i.e. 5-fluoro-3-(difluoromethyl)-5-fluoro-1-methyl-1H-pyrazole-4-carboxylic acid.

### 2. Description of the Prior Art

Succinate dehydrogenase inhibitors (SDHIs) are a well-known class of agrochemicals (Fungicides) for disease control to protect cereals as well as fruit and vegetables for more than a decade. For example, well-known marketed representative compounds are Isoflucypram, Bixafen, Fluxapyroxad, Fluindapyr, Sedaxane, Isopyrazam and Benzovindifupyr. The manufacture or synthesis of such compounds strongly depends on fluorinated pyrazoles as key starting materials (key building blocks), and on environmentally friendly and industrially suitable processes to provide said fluorinated pyrazoles starting materials, and which in turn then allow for the manufacture or synthesis in environmentally friendly and industrially suitable processes as well.

A recent overview of scientific developments and improvements in general and especially for the compound Isoflucypram is given in scientific literature, e.g., in the journal Pest Manag Sci 76 (2020), page 3340-3347 (DOI: https://doi.org/10.1002/ps.5951).

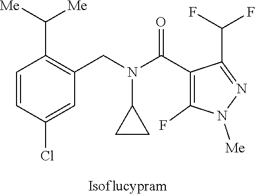 [SMILES: CC(C)c1ccc(Cl)cc1CN(C(=O)c1c(C(F)F)nn(C)c1F)C1CC1]

Me=methyl (—CH₃)

The provision of the pyrazole moiety of the targeted molecule structures incurs most of the synthetic challenges; hence, chemical improvements can be made at different points of the multistage synthesis routes to the pyrazole building blocks, such as the fluorination, the cyclization and potential further functionalization of the pyrazole moiety of the targeted molecules.

Related to the synthesis of the fluorinated pyrazole building block, a kind of review was already published in Organic Process Research & Development (OPRD 2014, 18, 1055-1059); and for the improvement of the cyclization step, e.g., in WO2020/093715. A procedure with waterfree hydrazines is described, which due to avoiding water and applying a special reactor design, increases the overall yields, for the reason of avoiding formation of the regio-isomers, and avoiding the necessity of the treatment of huge amounts of toxic waste water.

Regarding the compound Isoflucypram, it's the first time that a 5-fluoro atom substituted difluoromethyl pyrazole moiety is used which adds another challenge to assure a large-scale industrial and environmentally friendly synthesis of the pyrazole building block.

In WO2011/061205 the synthesis of the representative 5-fluoro-difluoromethyl pyrazole building block is starting from the compound ethyl difluoro acetoacetate (EDFAA), and ivolves an initial cyclization reaction with monomethyl hydrazine (MMH), followed by chlorination in 5-position with POCl₃ and formylation with DMF to give the 3-(difluoromethyl)-5-fluoro-1-methyl-1H-pyrazole-4-carboxaldehyde as outlined in the Scheme below. The principle of this synthesis route for pyrazoles and isoxazoles was already described by Kumiai in WO03/000686 and WO2004/014138 for the CF₃-derivative.

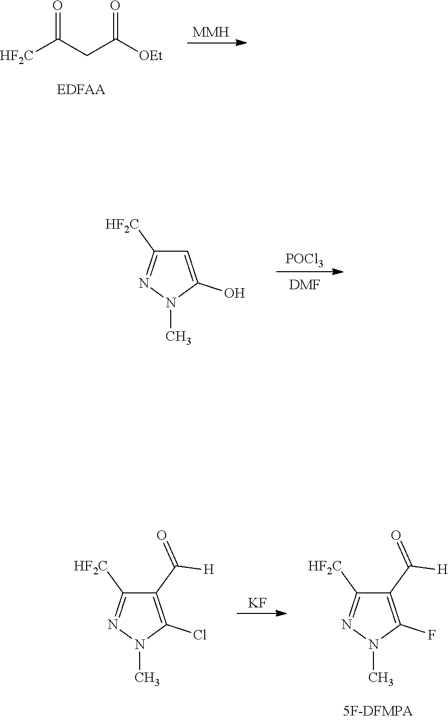

Et=ethyl (—CH₂CH₃); MMH=methyl hydrazine; DMF=dimethylformamide

The multiple drawbacks of this synthesis route are that starting compound EDFAA is quite unstable, MMH must be used as a solution in water (water is only for safety reasons), the resulting aldehyde chlorinated in 5-position is another quite sensitive intermediate, and a quite crude (“dirty”) Halex reaction with KF as fluorinating agent is required to finally give the targeted pyrazole fluorinated in 5-position (5F-DFMPA). The major drawbacks of an industrially performed Halex reaction are the need of large amounts of solvent, the formation of KCl (even contaminated with toxic material) which has to be separated off from the 5F-DFMPA product by hydrolysis, thus causing formation of toxic waste water. Overall, by this synthesis route, toxic waste water is formed at multiple stages in the synthesis, e.g., in the cyclization step, the chlorination step and also in the fluorination step. This compromises the applicability of such synthesis route for any industrial scale manufacturing.

Due to the instabilities and drawbacks described above in the synthesis route involving the CF₂H-group, in the patent publication WO2015/110493, a modification of the above synthesis route is disclosed, instead involving a CF₂Cl-group as more stable moiety in the starting material. In the last step the CF₂H-group is formed out of the CF₂Cl-group by a reductive dehalogenation with hydrogen over 5% Pd on CaCO₃ catalyst. Another patent publication (WO2011/131615) is dedicated to the preparation of 3-(difluoromethyl)-5-halo-1-methyl-1H-pyrazole-4-carboxylic acid halogenides, but uses also two steps, such as chlorination followed by fluorination and using also the crude (“dirty”) Halex reaction. Besides disadvantageous hydrolysis in the synthesis route as above, here also disadvantageous usage of Sulfolan as solvent and other solvents, which solvents are quite difficult or even not removable, are required to be used in Halex reactions. All these drawbacks of such synthesis route, again, compromise very much its applicability for any industrial scale manufacturing.

It is an objective of the present invention to overcome the drawbacks of the prior art processes for the synthesis of fluorinated pyrazole building blocks, e.g., in particular of the 5-fluoro-difluoromethyl pyrazole building block. It is another objective to provide a process for the synthesis of fluorinated pyrazole building blocks, e.g., in particular of the 5-fluoro-difluoromethyl pyrazole building block, which is suitable for a large-scale industrial and environmentally friendly synthesis of the fluorinated pyrazole building blocks, e.g., in particular of the 5-fluoro-difluoromethyl pyrazole building block. It is still another objective to provide a process for the synthesis of fluorinated pyrazole building blocks in high

### SUMMARY OF THE INVENTION

The object of the invention is solved as defined in the claims, and described herein after in detail.

The invention provides a new process for the synthesis of 5-fluoro-3-(difluoromethyl)-5-fluoro-1-methyl-1H-pyrazole-4-carboxylic acid derivatives and the free acid thereof, involving a direct fluorination reaction with a fluorination gas comprising or consisting of elemental fluorine (F₂), in a reactor which is resistant to elemental fluorine (F₂) and hydrogen fluoride (HF), and wherein in the process as a starting material a difluoromethyl-pyrazole compound dissolved in an inert solvent is subjected to the direct fluorination reaction.

The term “derivative” in the context of the current invention means a carboxylic acid derivative, i.e., a 5-fluoro-3-(difluoromethyl)-5-fluoro-1-methyl-1H-pyrazole-4-carboxylic acid derivatives is meant to be, a derivative of the 5-fluoro-3-(difluoro-methyl)-5-fluoro-1-methyl-1H-pyrazole-4-carboxylic acid (i.e., of the free acid) wherein the carboxylic acid group is derivatized. Typically known carboxylic acid derivatives are the carboxylic acid halogenides, carboxylic acid esters, and carboxylic acid amides. Typically known carboxylic acid halogenides (Hal) are the carboxylic acid fluoride (Hal=F), carboxylic acid chloride (Hal=Cl), or carboxylic acid bromide (Hal=Br). According to the process of the current invention, in particular the carboxylic acid fluoride (Hal=F) is preferred among the carboxylic acid halogenides (Hal). Typical and normally preferred carboxylic acid esters are, e.g., carboxylic acid methyl ester and carboxylic acid ethyl ester, and carboxylic acid benzyl ester. Carboxylic acid esters made by the process of the current invention, if desired, can be converted into carboxylic acid halogenides (Hal) by conventional methods know to the person skilled in the field. Virtually any solvent which is resistant to elemental fluorine (F₂) and hydrogen fluoride (HF) can be used as solvent (i.e., as inert solvent) in the direct fluorination reaction or process of the current invention. In context of the invention the term “inert solvent” means an inert organic solvent and/hydrogen fluoride (HF); wherein the inert solvent has good inertness against elemental fluorine (F₂) and against hydrogen fluoride (HF). The inert solvent shall also have a good solubility for the starting materials and (raw) product materials.

The use of an inert solvent is advantageous because starting materials used in the current invention are either high-boiling, oily and viscous substances (e.g., the aldehyde shown in Scheme 1b below has a boiling point of 260° C.; e.g., the acid chloride shown in Scheme 1a below has a boiling point of 276° C.) or are solids (e.g. the ethyl ester shown in Scheme 1a below). Thus, although theoretically possible to use oily starting materials without an inert solvent, the use of an inert solvent is preferred.

Surprisingly, in contrast to the prior art where in F₂-gas fluorination reactions in general a low selectivity regarding the fluorination site is observed (e.g., whenever next to the desired C—H fluorination site further C—H-bonds are present), the current invention circumvents the as mentioned above for the prior art, especially drawbacks related to a crude (“dirty”) (“dirty”) chlorination (e.g., Halex reaction) and/or related to a crude (“dirty”) fluorination (e.g., with KF as fluorinating agent). As stated before, both prior art reactions disadvantageously cause a lot of waste water.

The benefits of the present invention are surprisingly achieved by substituting the previously mentioned two crude (“dirty”) reaction steps, e.g., the Halex chlorination and the fluorination with KF, for using a direct fluorination reaction with a fluorination gas comprising or consisting of elemental fluorine (F₂), and by using as a starting material a difluoromethyl-pyrazole compound dissolved in an inert solvent.

The acid derivative of 5-fluoro-3-(difluoromethyl)-5-fluoro-1-methyl-1H-pyrazole-4-carboxylic acid in the context of the present invention is a 5-fluoro-difluoromethyl-pyrazole compound having the following formula (I),

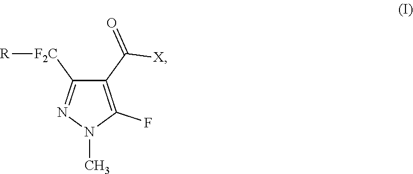

wherein
   R represents H (hydrogen atom), Cl (chlorine atom), Br (bromine atom) or F (fluorine atom), and
   X represents F (fluorine atom), or a —O—R¹ group wherein R¹ represents a C1-C4-alkyl group, a benzyl group or a substituted benzyl group.

Accordingly, in one aspect of the invention, the invention relates to a process for the manufacture of a 5-fluoro-difluoromethyl-pyrazole compound having the above formula (I), wherein R has the meaning as defined above and X has the meaning as defined above, and wherein in the process as a starting material a difluoromethyl-pyrazole compound of formula (II),

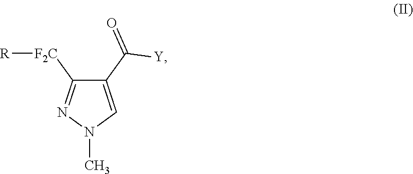

wherein
   R represents H (hydrogen atom), Cl (chlorine atom), Br (bromine atom), F (fluorine atom), and
   Y represents H (hydrogen atom), Cl (chlorine atom), or a —O—R¹ group wherein R¹ represents a C1-C4-alkyl group, a benzyl group or a substituted benzyl group;
dissolved in an inert solvent is subjected to a direct fluorination reaction with a fluorination gas comprising or consisting of elemental fluorine (F₂), in a reactor which is resistant to elemental fluorine (F₂) and hydrogen fluoride,
to form a reaction mixture containing the 5-fluoro-difluoromethyl-pyrazole compound having the formula (I),
wherein R has the meaning as defined here above and with the provisos that
   (i) X in formula (I) is F (fluorine atom) if Y formula (II) is H (hydrogen atom) or Cl (chlorine atom) or Br (bromine atom), and
   (ii) X in formula (I) is a —O—R¹ group if Y formula (II) is a —O—R¹ group, and wherein R¹ is as defined here above for X and Y, and wherein R¹ in X has the same meaning as in Y;
and optionally isolating from the reaction mixture and/or purifying, to yield the isolated and/or purified 5-fluoro-difluoromethyl-pyrazole compound having the formula (I).

The 5-fluoro-3-(difluoromethyl)-5-fluoro-1-methyl-1H-pyrazole-4-carboxylic acid in the context of the present invention is a 5-fluoro-difluoromethyl-pyrazole compound having following the formula (Ia),

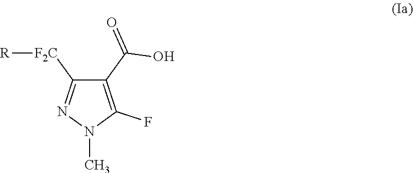

wherein
   R represents H (hydrogen atom), Cl (chlorine atom), Br (bromine atom), F (fluorine atom).

The 5-fluoro-3-(difluoromethyl)-5-fluoro-1-methyl-1H-pyrazole-4-carboxylic acid having the above formula (Ia), can be obtained from its carboxylic acid derivatives such as a 5-fluoro-difluoromethyl-pyrazole compound having the above formula (I), wherein R has the meaning as defined above and X has the meaning as defined above, in that the —C(═O)—X group in the acid derivative of formula (I) is converted to the carboxylic acid group —C(═O)—OH), by a conventional reaction known to a person skilled in the art, by which the substituent X in a compound of formula (I) can be converted into a —OH group. Such conversion can be achieved, for example, by hydrolysis and/or saponification. If the substituent X is a benzyl or substituted benzyl, a catalytic conversion into the free acid is also possible, e.g., by hydrogenation over a Pt, Pd, Rh, or other noble metal catalyst. However, hydrolysis and/or saponification is also preferred for the acid derivatives wherein the substituent X is a benzyl or substituted benzyl.

Accordingly, in another aspect of the invention, the invention also relates a process for the manufacture of a 5-fluoro-3-(difluoromethyl)-5-fluoro-1-methyl-1H-pyrazole-4-carboxylic acid having the above formula (Ia),

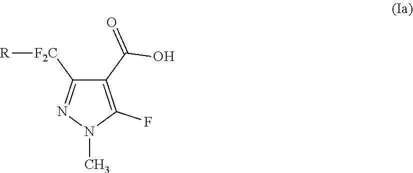

wherein
   R represents H (hydrogen atom), Cl (chlorine atom), Br (bromine atom), F (fluorine atom),
      and wherein in the process as a starting material a 5-fluoro-difluoromethyl-pyrazole compound having the formula (I),

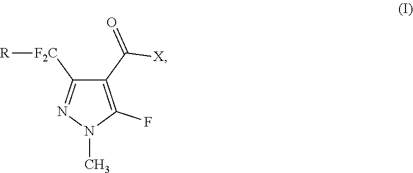

wherein
   R represents H (hydrogen atom), Cl (chlorine atom), Br (bromine atom) or F (fluorine atom), and
   X represents F (fluorine atom), or a —O—R¹ group wherein R¹ represents a C1-C4-alkyl group, a benzyl group or a substituted benzyl group.
(i) is subjected to a hydrolysis and/or saponification reaction, or
(ii) in case that in the —O—R¹ group the substituent R¹ represents a benzyl group or a substituted benzyl group, is subjected to a hydrolysis and/or saponification reaction, or alternatively to a (mild) catalytic hydrogenation,
to convert the substituent X into a —OH group, and to yield the acid compound 5-fluoro-3-(difluoromethyl)-5-fluoro-1-methyl-1H-pyrazole-4-carboxylic acid having the formula (Ia),
and optionally isolating and/or purifying, to yield the isolated and/or purified acid compound 5-fluoro-3-(difluoromethyl)-5-fluoro-1-methyl-1H-pyrazole-4-carboxylic acid compound having the formula (Ia).

In still a further aspect of the invention, more importantly, the acid derivative of 5-fluoro-3-(difluoromethyl)-5-fluoro-1-methyl-1H-pyrazole-4-carboxylic acid prepared according to the present invention, i.e., a 5-fluoro-difluoromethyl-pyrazole compound having the following formula (I) as defined herein above, is useful for preparing active ingredients for agrochemicals (e.g., fungicides), for example, of succinate dehydrogenase inhibitors (SDHIs), which are used for disease control to protect cereals as well as fruit and vegetables for more than a decade. The manufacture or synthesis of such succinate dehydrogenase inhibitors (SDHIs) strongly depends on fluorinated pyrazoles as key starting materials (key building blocks),

Thus, the acid derivative of 5-fluoro-3-(difluoromethyl)-5-fluoro-1-methyl-1H-pyrazole-4-carboxylic acid prepared according to the present invention, i.e., a 5-fluoro-difluoromethyl-pyrazole compound having the following formula (I) as defined herein above, is useful for preparing active ingredients (“AI”) of said agrochemicals (e.g., fungicides), for example, for such like Isoflucypram, Bixafen, Fluxapyroxad, Fluindapyr, Sedaxane, Isopyrazam and Benzovindifupyr. The manufacture or synthesis of such compounds strongly depends on fluorinated pyrazoles as key starting materials (key building blocks), as shown for example, hereinafter for the preparation of Isoflucypram:

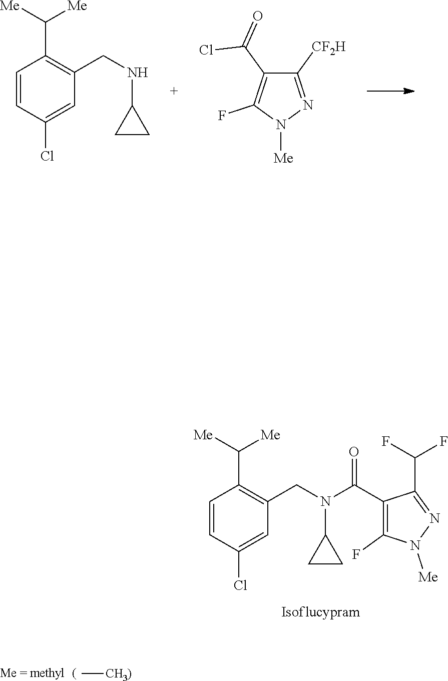

The before said active ingredients (“AI”) produced from pyrazole carboxylic acid derivatives (i.e., the acid derivative of 5-fluoro-3-(difluoromethyl)-5-fluoro-1-methyl-1H-pyrazole-4-carboxylic acid prepared according to the present invention, i.e., a 5-fluoro-difluoromethyl-pyrazole compound having the following formula (I) as defined herein above) are all amide compounds, like shown by the above representative formula of Isoflucypram.

Therefore, it is preferred to prepare the before said active ingredients (“AI”) of agrochemicals (e.g., fungicides), for example, Isoflucypram, Bixafen, Fluxapyroxad, Fluindapyr, Sedaxane, Isopyrazam and Benzovindifupyr, from the from the corresponding carboxylic acid halides, e.g., from the carboxylic acid fluoride having the formula (I) wherein R is defined as above and X is F (fluorine atom) or from the corresponding carboxylic acid chloride (Cl instead of X═F), of the acid derivative of a 5-fluoro-difluoromethyl-pyrazole compound having the following formula (I) as defined herein above.

It is more preferred to prepare the before said active ingredients (“AI”) of agrochemicals (e.g., fungicides), for example, Isoflucypram, Bixafen, Fluxapyroxad, Fluindapyr, Sedaxane, Isopyrazam and Benzovindifupyr, from the from the corresponding carboxylic acid chloride (Cl instead of X═F). Said corresponding acid chloride, having the following formula (Ib), can be prepared from the acid derivative of a 5-fluoro-difluoromethyl-pyrazole compound having the following formula (I) as defined herein above, or from the 5-fluoro-3-(difluoromethyl)-5-fluoro-1-methyl-1H-pyrazole-4-carboxylic acid having the above formula (Ia), by methods known to the person skilled in the art.

The 5-fluoro-3-(difluoromethyl)-5-fluoro-1-methyl-1H-pyrazole-4-carboxylic acid chloride, which can be prepared from the pyrazole compounds of formula (I) or of formula (Ia), have the following formula (Ib),

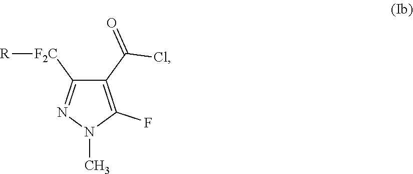

wherein
   R represents H (hydrogen atom), Cl (chlorine atom), Br (bromine atom) or F (fluorine atom).

As mentioned, the active ingredients (AI) produced from the pyrazole carboxylic acid derivatives described above are all amide compounds, i.e. these are preferably produced from the carboxylic acid halides, in particular from the carboxylic acid chlorides, by reaction with the corresponding precursors containing substituted amine groups or NH-2-groups (Schotten-Baumann process), see for example, EP2920151. Of course, the carboxylic acid fluorides also work, but partially liquid HF (b.p.: 21° C.) is somewhat more difficult to separate off and is more dangerous than HCl with a b.p. of −80° C. In older publications, a base is still required as an HCl trap (Schotten-Baumann process), since otherwise, i.e., without a base, the HCl salts of the active ingredients (AI) are obtained. The said pyrazole-based active ingredients (AI) can also be produced from the corresponding pyrazole carboxylic esters, for example in WO 2016016298 (Solvay).

### Definitions

Direct Fluorination: Introducing one or more fluorine atoms into a compound by chemically reacting a starting compound with elemental fluorine (F₂) such that one or more fluorine atoms are covalently bound into the reacted starting compound.

The term “liquid medium” may mean a solvent which inert to fluorination under the reaction conditions of the direct fluorination, in which the starting compound and/or fluorinated target compound may be dissolved, and/or the starting compound itself may be a liquid serving itself as liquid medium, and in which the fluorinated target compound may be dissolved if it is not a liquid, or if it is a liquid may also serve as the liquid medium.

The numerical ranges disclosed herein include all values from, and including, the lower and upper value. For ranges containing explicit values (e.g., 1 to 7), any subrange between any two explicit values is included (e.g., 1 to 2; 2 to 6; 5 to 7; 3 to 7; 5 to 6; etc.).

The terms “comprising,” “including,” “having,” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term, “consisting essentially of” excludes from the scope of any succeeding recitation any other component, step, or procedure, excepting those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa.

The term “vol.-%” as used herein means “% by volume”. Unless otherwise stated, all percentages (%) as used herein denote “vol.-%” or “% by volume”, respectively.

For example, the use of the term “essentially”, in referring to a fluorination gas consisting essentially of F₂-gas as it directly comes out of the F₂-electrolysis reactors (fluorine cells), means that providing such F₂-gas does not involve major purification and/or providing another gas, e.g., an inert gas, separate and/or in admixture in amounts and/or under conditions that would be sufficient to provide a change in the composition of an F₂-gas as produced in and as it is withdrawn as gaseous product from F₂-electrolysis reactors (fluorine cells) of more than about ±5% by volume, or preferably of more than about ±3% by volume. Accordingly, such a fluorination gas consisting essentially of F₂-gas as it directly comes out of the F₂-electrolysis reactors (fluorine cells) is meant to comprise elemental fluorine (F₂) in a concentration of at least about 92% by volume, or preferably of at least about 95% by volume. Especially, such a fluorination gas consisting essentially of F₂-gas as it directly comes out of the F₂-electrolysis reactors (fluorine cells) may comprise elemental fluorine (F₂) in a concentration in a range of about 92-100% by volume, or preferably in a range of about 95-100% by volume, or more preferably in a range of in a range of about 92-99% by volume, or preferably in a range of about 95-99% by volume, or in a range of in a range of about 92 to about 97% by volume, or preferably in a range of about 95 to about 97% by volume.

yield and high regio-selectivity.

### BRIEF DESCRIPTION OF THE DRAWINGS

FIG. 1 shows Direct Fluorination using a gas scrubber system.

FIG. 2 shows Continuous fluorination in a one or several microreactor (in series) system.

FIG. 3 shows Continuous fluorination in a coil reactor system.

### DETAILED DESCRIPTION OF THE INVENTION

The invention provides a new process for the synthesis of 5-fluoro-3-(difluoromethyl)-5-fluoro-1-methyl-1H-pyrazole-4-carboxylic acid derivatives and the free acid thereof, involving a direct fluorination reaction with a fluorination gas comprising or consisting of elemental fluorine (F₂), in a reactor which is resistant to elemental fluorine (F₂) and hydrogen fluoride (1F), and wherein in the process as a starting material a difluoromethyl-pyrazole compound dissolved in an inert solvent is subjected to the direct fluorination reaction. The new process of the invention is suitable for the synthesis of 5-fluoro-3-(difluoromethyl)-5-fluoro-1-methyl-1H-pyrazole-4-carboxylic acid derivatives, i.e., derivatives of 5-fluoro-3-(difluoromethyl)-5-fluoro-1-methyl-1H-pyrazole-4-carboxylic acid (the free acid) wherein the carboxylic acid group is derivatized, and the free acid thereof, i.e. for the synthesis of 5-fluoro-3-(difluoromethyl)-5-fluoro-1-methyl-1H-pyrazole-4-carboxylic acid.

The process of the invention is performed in an inert solvent, e.g., starting material compounds and resulting products are dissolved in an inert solvent. Virtually any solvent which is resistant to elemental fluorine (F₂) and hydrogen fluoride (HF) can be used as solvent (i.e., as inert solvent) in the direct fluorination reaction or process of the current invention. In context of the invention the term “inert solvent” means an inert organic solvent and/hydrogen fluoride (HF); wherein the inert solvent has good inertness against elemental fluorine (F₂) and against hydrogen fluoride (HF).

The inert solvent shall also have a good solubility for the starting materials and (raw) product materials. Particular examples of inert solvents are, next to hydrogen fluoride (HF), e.g., anhydrous hydrogen fluoride (anhydrous HF), are formic acid, trifluoroacetic acid, acetonitrile.

For example, an inert organic solvent suitable for the process of the invention is, e.g., acetonitrile (CH₃CN), formic acid, and trifluoroacetic acid, or are fully or partially fluorinated alkanes like pentafluorobutane (365mfc), linear or cyclic partially or fully fluorinated ethers like CF₃—CH₂—OCHF₂ (E245) or perhalogenated ethers like e.g. CF₃—O—CF₂—CCl₃ (b.p. 87° C.), or octafluorotetrahydrofurane. Linked to inertness against elemental fluorine (F₂), fully fluorinated (or at least fully halogenated) solvents, for example, such as perhalogenated compounds like CFCl₃ (to be used under higher pressures only), CF₂Cl—CFCl₂ (113, has a preferred boiling point of 48° C.) are also suitable inert organic solvents.

As mentioned before, hydrogen fluoride (HF), e.g., anhydrous hydrogen fluoride (anhydrous HF), is also a suitable inert (inorganic) solvent for the process of the invention. The inert solvent can also be Olah's reagent (pyridine/HF). If the inert solvent is Olah's reagent (pyridine/HF), this allows easier preparation of starting material solution but due to pyridine, this inert solvent is more difficult to remove out of product after reaction),

Accordingly, in the process of the invention the inert solvent is selected from the group consisting of hydrogen fluoride (HF), anhydrous hydrogen fluoride (anhydrous HF), Olah's reagent (pyridine/HF), acetonitrile (CH₃CN), formic acid, and trifluoroacetic acid, fluorinated alkane pentafluorobutane (365mfc), fluorinated ether CF₃—CH₂—OCHF₂ (E245, perhalogenated ether CF₃—O—CF₂—CCl₃, octafluorotetrahydrofurane, perhalogenated compound CFCl₃, and perhalogenated compound CF₂Cl—CFCl₂ (113). All these inert solvents have good solubility for the materials, and inertness against elemental fluorine (F₂) and hydrogen fluoride (HF).

Some particular examples of 5-fluoro-3-(difluoromethyl)-5-fluoro-1-methyl-1H-pyrazole-4-carboxylic acid derivatives which can be prepared according to the process of the current invention are the following compounds:
3-(difluoromethyl)-5-fluoro-1-methyl-1H-pyrazole-4-carboxylic acid fluoride (5F-DFMPAF), also known under the alternative name 3-(difluoromethyl)-5-fluoro-1-methyl-1H-pyrazole-4-carbonyl fluoride (see, e.g., also Example 1);
3-(difluoromethyl)-5-fluoro-1-methyl-1H-pyrazole-4-carboxylic acid ethylester (5F-DFMP), (see, e.g., also Example 2); or analogously the 3-(chlorodifluoromethyl)-5-fluoro-1-methyl-1H-pyrazole-4-carboxylic acid methylester, (e.g., preparation analogously to Example 2)
3-(chlorodifluoromethyl)-5-fluoro-1-methyl-1H-pyrazole-4-carboxylic acid ethylester (5F-CDFMP), (see, e.g., also Example 3); or analogously the 3-(chlorodifluoromethyl)-5-fluoro-1-methyl-1H-pyrazole-4-carboxylic acid methylester, (e.g., preparation analogously to Example 3).

The synthesis of said compounds according to the process of the current invention is exemplified in the following Reaction Schemes 1a to 1c.

If an acid chloride compound of formula (II) (R═Cl) is the starting material in the process of the invention, the resulting product is the corresponding acid fluoride compound, fluorinated in the 5-position of the pyrazole ring, as the HF formed by fluorination will result in a Cl/F-exchange reaction to form the corresponding acid fluoride compound (i.e., carbonyl fluoride compound). See, for example Reaction Scheme 1a below.

If an aldehyde compound of formula (II) (R═H) is the starting material in the process of the invention, the resulting product is also the corresponding acid fluoride compound, fluorinated in the 5-position of the pyrazole ring, as the —CH═O group (aldehyde group) will also be fluorinated to form the corresponding acid fluoride group (i.e., carbonyl fluoride group). See, for example Reaction Scheme 1b below.

If an ester compound of formula (II) (R=—O—R¹ wherein R¹ is defined as given herein after for formula (II)) is the starting material in the process of the invention, the resulting product is the corresponding ester compound, fluorinated in the 5-position of the pyrazole ring, but the ester group (R=—O—R¹) is not affected by fluorination, and thus is preserved in the product. See, for example Reaction Scheme 1c below.

Reaction Schemes 1a to 1c:

Meanings in the Scheme: R═H, Cl, F; Et=ethyl (—CH₂CH₃).

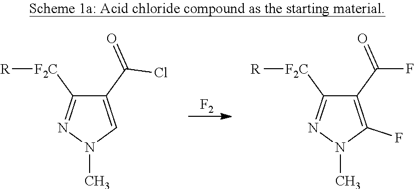

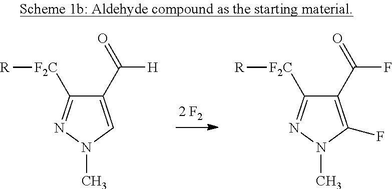

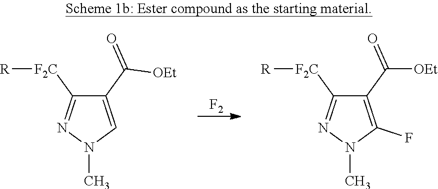

The present invention distinguish over the prior art processes in that the manufacture of the targeted 5-fluoro-3-(difluoromethyl)-5-fluoro-1-methyl-1H-pyrazole-4-carboxylic acid derivative compound is achieved by a process involving a more efficient fluorination reaction of particular starting material compounds, which are fluorinated with elemental fluorine (F₂).

Thereby, the present invention overcomes deficiencies or disadvantages, respectively, of the prior art processes, and especially satisfies the prior high demand of establishing a more efficient process, and in particular an industrial process, and more preferably a large-scale industrial process, for the manufacture of the targeted 5-fluoro-3-(difluoromethyl)-5-fluoro-1-methyl-1H-pyrazole-4-carboxylic acid derivative compound.

In this regard, the invention advantageously also provides for large-scale and/or industrial production processes without forming large amounts of waste water and non-recyclable salts which can contain very toxic particles, avoiding the formation of salts that cannot be economically recycled.

The fluorination reaction can be done in a batch reactor or even continuously in a series of STRs, plug flow or in so called microreactor or coil reactor. For work up, the equimolar formed HF can be removed out of the final solution after fluorination by applying a slight vacuum or using a small inert gas stream into a cooling trap to condense the HF or at least at part of it into an efficient loop (scrubber) system. In a batch system using a state of the art STR, only F₂ diluted with inert gas is economically practicable (an inert gas helps to avoid hot spots). In a counter-current system, microreactor system and coil reactor system, high concentrated F₂, optionally directly out of an F₂ electrolysis cell gives good yields and is applicable.

In the processes of the invention, a turbulent reaction state is preferred, for example, for allowing high production capacity and better selectivity. But, the skilled person will understand that turbulence is not intended to limit the process of the invention, especially as chemistry-wise turbulence is not mandatory for the reaction systems, such as counter-current reactor system, microreactor system, or coil reactor system, respectively.

Especially a microreactor system works the better the less inert gases are present (can form bubbles in the channels which inhibit heat transfer/heat exchanger efficiency).

In one aspect the invention relates to a process for the manufacture of a 5-fluoro-difluoromethyl-pyrazole compound having the formula (I),

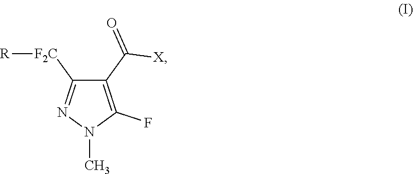

wherein
   R represents H (hydrogen atom), Cl (chlorine atom), Br (bromine atom) or F (fluorine atom), and
   X represents F (fluorine atom), or a —O—R¹ group wherein R¹ represents a C1-C4-alkyl group, a benzyl group or a substituted benzyl group;
wherein in the process as a starting material a difluoromethyl-pyrazole compound of formula (II),

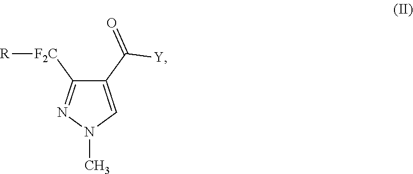

wherein
   R represents H (hydrogen atom), Cl (chlorine atom), Br (bromine atom), F (fluorine atom), and
   Y represents H (hydrogen atom), Cl (chlorine atom), or a —O—R¹ group wherein R¹ represents a C1-C4-alkyl group, a benzyl group or a substituted benzyl group;
dissolved in an inert solvent is subjected to a direct fluorination reaction with a fluorination gas comprising or consisting of elemental fluorine (F₂), in a reactor which is resistant to elemental fluorine (F₂) and hydrogen fluoride, to form a reaction mixture containing the 5-fluoro-difluoromethyl-pyrazole compound having the formula (I),
wherein R has the meaning as defined here above and with the provisos that
   (i) X in formula (I) is F (fluorine atom) if Y formula (II) is H (hydrogen atom) or Cl (chlorine atom) or Br (bromine atom), and
   (ii) X in formula (I) is a —O—R¹ group if Y formula (II) is a —O—R¹ group, and wherein R¹ is as defined here above for X and Y, and wherein R¹ in X has the same meaning as in Y;
and optionally isolating from the reaction mixture and/or purifying, to yield the isolated and/or purified 5-fluoro-difluoromethyl-pyrazole compound having the formula (I).

In another aspect the invention relates to a process for the manufacture of a 5-fluoro-difluoromethyl-pyrazole compound having the formula (I), wherein
R represents H (hydrogen atom), Cl (chlorine atom) or F (fluorine atom), and
X represents F (fluorine atom);
wherein in the process as a starting material a difluoromethyl-pyrazole compound of formula (II), wherein
   R represents H (hydrogen atom), Cl (chlorine atom) or F (fluorine atom), and
   Y represents H (hydrogen atom), Cl (chlorine atom);
dissolved in an inert solvent is subjected to a direct fluorination reaction with a fluorination gas comprising or consisting of elemental fluorine (F₂), in a reactor which is resistant to elemental fluorine (F₂) and hydrogen fluoride,
to form a reaction mixture containing the 5-fluoro-difluoromethyl-pyrazole compound having the formula (I), and optionally isolating from the reaction mixture and/or purifying, to yield the isolated and/or purified 5-fluoro-difluoromethyl-pyrazole compound having the formula (I).

In a further aspect the invention relates to a process for the manufacture of a 5-fluoro-difluoromethyl-pyrazole compound having the formula (I), wherein
R represents H (hydrogen atom), Cl (chlorine atom) or F (fluorine atom), and
X represents a —O—R¹ group wherein R¹ represents a C1-C4-alkyl group, a benzyl group or a substituted benzyl group, and preferably wherein R¹ represents a C1-C4-alkyl group;
wherein in the process as a starting material a difluoromethyl-pyrazole compound of formula (II), wherein
   R represents H (hydrogen atom), Cl (chlorine atom) or F (fluorine atom), and
   Y represents a —O—R¹ group wherein R¹ represents a C1-C4-alkyl group, a benzyl group or a substituted benzyl group, and preferably wherein R¹ represents a C1-C4-alkyl group;
dissolved in an inert solvent is subjected to a direct fluorination reaction with a fluorination gas comprising or consisting of elemental fluorine (F₂), in a reactor which is resistant to elemental fluorine (F₂) and hydrogen fluoride,
to form a reaction mixture containing the 5-fluoro-difluoromethyl-pyrazole compound having the formula (I), and optionally isolating from the reaction mixture and/or purifying, to yield the isolated and/or purified 5-fluoro-difluoromethyl-pyrazole compound having the formula (I).

The invention relates to a direct fluorination process, as mentioned herein above, wherein the direct fluorination reaction is carried out until no exothermic activity is observed.

The invention relates to a direct fluorination process, as mentioned herein above, wherein the direct fluorination reaction is carried out until no exothermic activity is observed in the reaction mixture.

The invention relates to a direct fluorination process, as mentioned herein above, wherein the direct fluorination reaction is carried out at a temperature which does not exceed a temperature of about 55° C., preferably does not exceed a temperature of about 50° C., more preferably does not exceed a temperature of about 45° C., even more preferably does not exceed a temperature of about 40° C., in the reaction mixture.

The invention relates to a direct fluorination process, as mentioned herein above, wherein the process is carried out such that HF (hydrogen fluoride) formed in the direct fluorination reaction is eliminated from the reaction mixture by purging the reaction mixture with an inert gas stream until no HF (hydrogen fluoride) is detected in the inert gas stream after it has passed through the reaction mixture.

The invention relates to a direct fluorination process, as mentioned herein above, wherein for isolating from the reaction mixture and/or purifying, the reaction mixture is subjected to one or more recrystallization, thereby to yield the isolated and/or purified 5-fluoro-difluoromethyl-pyrazole compound having the formula (I).

The invention relates to a direct fluorination process, as mentioned herein above, wherein for isolating from the reaction mixture and/or purifying, the reaction mixture is subjected to evaporating the inert solvent under vacuum from the reaction mixture, thereby to obtain as evaporation residue the isolated 5-fluoro-difluoromethyl-pyrazole compound having the formula (I), and optionally further purifying of the evaporation residue to yield the isolated and purified 5-fluoro-difluoromethyl-pyrazole compound having the formula (I).

The invention relates to a direct fluorination process, as mentioned herein above, wherein the further purifying of the evaporation residue comprises one or more recrystallization, thereby to yield the isolated and/or purified 5-fluoro-difluoromethyl-pyrazole compound having the formula (I).

The invention relates to a direct fluorination process, as mentioned herein above, wherein the elemental fluorine (F₂) is present in the fluorination gas of b) in a (“lower”) concentration in the range of up to about 20% by volume (vol.-%), or approximately about 20% by volume (vol.-%), each based on the total volume of the fluorination gas as 100% by volume.

The invention relates to a direct fluorination process, as mentioned herein above, wherein the elemental fluorine (F₂) is present in the fluorination gas of b) in a concentration in the “lower” range of from 0.1% by volume (vol.-%) up to about 20% by volume (vol.-%), in the range of from 0.5% by volume (vol.-%) up to about 20% by volume (vol.-%), in the range of from 1% by volume (vol.-%) up to about 20% by volume (vol.-%), in the range of from 5% by volume (vol.-%) up to about 20% by volume (vol.-%), in the range of from 10% by volume (vol.-%) up to about 20% by volume (vol.-%), in the range of from 15% by volume (vol.-%) up to about 20% by volume (vol.-%), or in a concentration of approximately about 20% by volume (vol.-%), each based on the total volume of the fluorination gas as 100% by volume.

The invention relates to a direct fluorination process, as mentioned herein above, wherein the elemental fluorine (F₂) is present in the fluorination gas of b) in a high concentration of at least about 15% by volume, in particular in a high concentration of at least about 20% by volume, preferably in a high concentration of at least about 25% by volume, further preferably of at least about 30% by volume, more preferably of at least about 35% by volume, even more preferably of at least about 45% by volume, each based on the total volume of the fluorination gas as 100% by volume.

The invention relates to a direct fluorination process, as mentioned herein above, wherein the fluorine (F₂) is present in the fluorination gas of b) in a high concentration within a range of from about 15-100% by volume, preferably within a range of from about 20-100% by volume, more preferably within a range of from about 25-100% by volume, still more preferably within a range of from about 30-100% by volume, even more preferably within a range of from about 35-100% by volume, an still more preferred within a range of from about 45-100% by volume, each based on the total volume of the fluorination gas as 100% by volume.

The elemental fluorine (F₂) used in the fluorination gas in a (“lower” or “higher”) concentration in the ranges given above in % by volume (vol.-%), based on the total volume of the fluorination gas as 100% by volume, The fluorination gas comprising the elemental fluorine (F₂) can directly or indirectly come from a fluorine cell or an “on-site” fluorine generator, and then one could have theoretically F₂ concentrations of up to 98% by volume. But practically spoken, some inert gas normally will be fed in, as the inert gas can also serve as a transport medium for effluent reaction products (e.g., such as HCl).

Therefore, preferably, the invention relates to a direct fluorination process, as mentioned herein above, wherein the fluorine (F₂) is present in the fluorination gas of b) in a high concentration within a range of from about 15-98% by volume, preferably within a range of from about 20-98% by volume, more preferably within a range of from about 25-98% by volume, still more preferably within a range of from about 30-98% by volume, even more preferably within a range of from about 35-98% by volume, an still more preferred within a range of from about 45-98% by volume, each based on the total volume of the fluorination gas as 100% by volume.

Furthermore, more preferably, the invention relates to a direct fluorination process, as mentioned herein above, wherein the fluorine (F₂) is present in the fluorination gas of b) in a high concentration within a range of from about 15-90% by volume, preferably within a range of from about 20-90% by volume, more preferably within a range of from about 25-90% by volume, still more preferably within a range of from about 30-90% by volume, even more preferably within a range of from about 35-90% by volume, an still more preferred within a range of from about 45-90% by volume, each based on the total volume of the fluorination gas as 100% by volume.

The invention relates to a direct fluorination process, as mentioned herein above, wherein the direct fluorination reaction is carried out in a (closed) column reactor, optionally either operated in a batch manner or operated in a continuous manner, wherein a solution of the starting material difluoromethyl-pyrazole compound dissolved in an inert solvent is circulated in a loop, while the fluorination gas comprising or consisting of elemental fluorine (F₂) in a high concentration is fed into the column reactor and is passed through the liquid medium to react with the starting compound to form a reaction mixture containing the 5-fluoro-difluoromethyl-pyrazole compound having the formula (I), and further circulating in a loop until the fluorination reaction is completed; preferably wherein the loop is operated with a circulation velocity of from 500 l/h to 5,000 l/h, more preferably of from 3,500 l/h to 4,500 l/h.

The invention relates to a direct fluorination process, as mentioned herein above, wherein the column reactor is equipped with at least one of the following:
(i) at least one cooler (system), at least one liquid reservoir, with inlet and outlet for, and containing as a liquid medium the starting material difluoromethyl-pyrazole compound dissolved in an inert solvent, and as the direct fluorination reaction proceeds also the reaction mixture containing the 5-fluoro-difluoromethyl-pyrazole compound having the formula (I);
(ii) a pump for pumping and circulating the liquid medium of (i) in the column reactor;
(iii) one or more (nozzle) jets, preferably wherein the one or more (nozzle) jets are placed at the top of the column reactor, for spraying the circulating liquid medium of (i) into the column reactor; or alternatively a perforated metal sheet placed at the top of the column reactor, for circulating the liquid medium of (i) into the column reactor, used together with a high-efficiency pump;
(iv) one or more feeding inlets for introducing the fluorination gas comprising or consisting of elemental fluorine (F₂) in a high concentration into the column reactor;
(v) optionally one or more sieves, preferably two sieves, preferably the one or more sieves placed at the bottom of the column reactor;
(vi) and at least one gas outlet equipped with a pressure valve, and at least one outlet for withdrawing the reaction mixture containing the 5-fluoro-difluoromethyl-pyrazole compound having the formula (I).

The invention relates to a direct fluorination process, as mentioned herein above, wherein column reactor is a packed bed tower reactor, preferably a packed bed tower reactor which is packed with fillers resistant to elemental fluorine (F₂) and hydrogen fluoride (HF), e.g. with Raschig fillers and/or metal fillers, more preferably wherein the packed bed tower reactor is a gas loop (scrubber) system (tower) which is packed with fillers resistant to elemental fluorine (F₂) and hydrogen fluoride (HF), e.g. HDPTFE Raschig fillers and/or metal fillers; the said fillers should have a diameter of not smaller than about 10 mm (not smaller that about 1 cm; e.g., not smaller than about 1±0.05 cm).).

The invention relates to a direct fluorination process, as mentioned herein above, wherein the direct fluorination reaction is carried out with a counter-current flow of the circulating

liquid medium of a) comprising or consisting of the starting compound and of the fluorination gas of b) fed into the column reactor and which fluorination gas of b) is comprising or consisting of elemental fluorine (F₂) in a high concentration.

The invention relates to a direct fluorination process, as mentioned herein above, wherein the direct fluorination reaction is carried out in a (closed) column reactor, operated in a continuous manner. The term “closed” is not meant to exclude safety valves, which may be present, or to exclude effluent means, for example, to provide (controlled) escape of inert gas, optionally together with at least a part or, alternatively, major or even substantial parts, if desired, of hydrogen fluoride (HF) gas. Of course, as stated above, if desired, at least a part or, alternatively, a major or even substantial part of hydrogen fluoride (HF) may be maintained in the reactor system as a solvent for the direct fluorination reaction.

The invention relates to a direct fluorination process, as mentioned herein above, wherein the direct fluorination reaction is carried out in a (closed) column reactor, which is made out of Hastelloy, preferably which is made out of Hastelloy C4.

TABLE 1
Chemical composition of Hastelloy C4 (nickel alloy).

| C | Si | Mn | P | S | Cr | Mo | Co | Fe | Ti |
|---|---|---|---|---|---|---|---|---|---|
| % | ≤% | ≤% | ≤% | ≤% | % | % | ≤% | % | % |
| 0- | 0- | 0-1.0 | 0- | 0- | 14.5- | 14.0- | 0-2.0 | 0-3 | 0-0.7 |
| 0.009 | 0.05 | | 0.02 | 0.01 | 17.5 | 17.0 | | | |

and nickel (Ni) as the remainder for adding up to 100 % metal alloy.

Next to preferred Hastelloy® C4 described here before, as stated already above, also Hastelloy® C22 is preferred, but has a slightly different composition than Hastelloy® C4. Hastelloy® C is an alloy represented by the formula NiCr21Mo14W, alternatively also known as “alloy 22” or “Hastelloy® C22. The said alloy is well known as a highly corrosion resistant nickel-chromium-molybdenum-tungsten alloy and has excellent resistance to oxidizing reducing and mixed acids. The said alloy is used in flue gas desulphurization plants, in the chemical industry, environmental protection systems, waste incineration plants, sewage plants. Apart from the before said example, in other embodiments of the invention, in general nickel-chromium-molybdenum-tungsten alloy from other manufactures, and as known to the skilled person, of course can be employed in the present invention. A typical chemical composition (all in weight-%) of such nickel-chromium-molybdenum-tungsten alloy is, each percentage based on the total alloy composition as 100%: Ni (nickel) as the main component (balance) of at least about 51.0%, e.g. in a range of from about 51.0% to about 63.0%; Cr (chromium) in a range of from about 20.0 to about 22.5%, Mo (molybdenum) in a range of from about 12.5 to about 14.5%, W (tungsten or wolfram, respectively) in a range of from about 2.5 to about 3.5%; and Fe (iron) in an amount of up to about 6.0%, e.g. in a range of from about 1.0% to about 6.0%, preferably in a range of from about 1.5% to about 6.0%, more preferably in a range of from about 2.0% to about 6.0%. Optionally, the percentage based on the total alloy composition as 100%, Co (cobalt) can be present in the alloy in an amount of up to about 2.5%, e.g. in a range of from about 0.1% to about 2.5%. Optionally, the percentage based on the total alloy composition as 100%, V (vanadium) can be present in the alloy in an amount of up to about 0.35%, e.g. in a range of from about 0.1% to about 0,35%. Also, the percentage based on the total alloy composition as 100%, optionally low amounts (i.e. ≤0.1%) of other element traces, e.g. independently of C (carbon), Si (silicon), Mn (manganese), P (phosphor), and/or S (sulfur). In such case of low amounts (i.e. ≤0.1%) of other elements, the said elements e.g. of C (carbon), Si (silicon), Mn (manganese), P (phosphor), and/or S (sulfur), the percentage based on the total alloy composition as 100%, each independently can be present in an amount of up to about 0.1%, e.g. each independently in a range of from about 0.01 to about 0.1%, preferably each independently in an amount of up to about 0.08%, e.g. each independently in a range of from about 0.01 to about 0.08%. For example, said elements e.g. of C (carbon), Si (silicon), Mn (manganese), P (phosphor), and/or S (sulfur), the percentage based on the total alloy composition as 100%, each independently can be present in an amount of, each value as an about value: C≤0.01%, Si≤0.08%, Mn≤0.05%, P≤0.015%, S≤0.02%. Normally, no traceable amounts of any of the following elements are found in the alloy compositions indicated above: Nb (niobium), Ti (titanium), Al (aluminum), Cu (copper), N (nitrogen), and Ce (cerium).

Hastelloy® C-276 alloy was the first wrought, nickel-chromium-molybdenum material to alleviate concerns over welding (by virtue of extremely low carbon and silicon contents). As such, it was widely accepted in the chemical process and associated industries, and now has a 50-year-old track record of proven performance in a vast number of corrosive chemicals. Like other nickel alloys, it is ductile, easy to form and weld, and possesses exceptional resistance to stress corrosion cracking in chloride-bearing solutions (a form of degradation to which the austenitic stainless steels are prone). With its high chromium and molybdenum contents, it is able to withstand both oxidizing and non-oxidizing acids, and exhibits outstanding resistance to pitting and crevice attack in the presence of chlorides and other halides. The nominal composition in weight-% is, based on the total composition as 100%: Ni (nickel) 57% (balance); Co (cobalt) 2.5% (max.); Cr (chromium) 16%; Mo (molybdenum) 16%; Fe (iron) 5%; W (tungsten or wolfram, respectively) 4%; further components in lower amounts can be Mn (manganese) up to 1% (max.); V (vanadium) up to 0.35% (max.); Si (silicon) up to 0.08% (max.); C (carbon) 0.01 (max.); Cu (copper) up to 0.5% (max.).

The invention relates to a direct fluorination process, as mentioned herein above, wherein the direct fluorination reaction is carried out in a coil reactor, operated in a continuous manner.

The invention relates to a direct fluorination process, as mentioned herein above, wherein the direct fluorination reaction is carried out in a coil reactor, which is made out of Hastelloy, preferably which is made out of Hastelloy C4.

The invention relates to a direct fluorination process, as mentioned herein above, wherein the direct fluorination reaction is carried out in a continuous flow reactor with upper lateral dimensions of about ≤5 mm, or of about ≤4 mm, operated in a continuous manner.

The invention relates to a direct fluorination process, as mentioned herein above, wherein the direct fluorination reaction is carried out in a microreactor, operated in a continuous manner.

The invention relates to a direct fluorination process, as mentioned herein above, wherein the direct fluorination reaction is carried out in as a continuous process in a microreactor under one or more of the following conditions:
flow rate: of from about 10 ml/h up to about 400 l/h;
temperature: of from about −20° C. up to about 150° C.;
pressure: of from about 1 bar (e.g. 1 atm abs.) up to about 50 bar;
residence time: of from about 1 second, preferably from about 1 minute, up to about 60 minutes.

The invention relates to a direct fluorination process, as mentioned herein above, wherein the microreactor is a SiC-microreactor.

Reactor Design and Direct Fluorination:

In this invention it was also found that the fluorination reaction can be carried out beneficially and preferably in special equipment and with special reactor design such as, e.g., a microreactor or a packed bed tower (preferably made of Hastelloy), especially a packed bed tower containing fillers, e.g., metal fillers (e.g. Hastelloy) or plastic fillers, preferably wherein the tower (e.g., made out of Hastelloy) is filled either with E-TFE or metal fillings (Hastelloy), for example each of about 10 mm diameter as available from Raschig (http://www.raschig.de/Fllkrper). The type of fillings is quite flexible, Raschigs Pall-Rings made out of Hastelloy can be used, and advantageously E-TFE-fillings, and especially HDPTFE-fillings.

In the said special equipment and with special reactor design such as, e.g., a microreactor or a packed bed tower (preferably made of Hastelloy), a fluorine gas with concentrations, as defined above and in the claims, can be used for chemical synthesis especially for the preparation of the fluorinated compound.

In a applying the present fluorination process it is possible to also perform chemistry with F₂ as it comes directly out of the F₂-electrolysis reactors (fluorine cells). A representative composition of fluorine gas produced by a fluorine cell is 97% F₂, up to 3% CF₄ (formed from damage of the electrodes), for example, traces of HF, NO₂, OF₂, COF₂, each % by volume and based on the total volume of the fluorine containing gas as 100% by volume.

In the fluorination gas the elemental fluorine (F₂) may be diluted by an inert gas. The inert gas then constitutes the substantial difference (e.g., there may be only minor quantities of by-products (e.g., CF₄) of no more than about 5% by volume, preferably of no more than about 3% by volume, and only traces impurities (e.g., such like HF, NO₂, OF₂, COF₂), in the fluorination gas).

An inert gas is a gas that does not undergo chemical reactions under a set of given conditions. The noble gases often do not react with many substances and were historically referred to as the inert gases. Inert gases are used generally to avoid unwanted chemical reactions degrading a sample. These undesirable chemical reactions are often oxidation and hydrolysis reactions with the oxygen and moisture in air.

Typical inert gases are noble gases, and the very common inert gas nitrogen (N₂). The noble gases (historically also the inert gases; sometimes referred to as aerogens) make up a group of chemical elements with similar properties; under standard conditions, they are all odorless, colorless, monatomic gases with very low chemical reactivity. The six noble gases that occur naturally are helium (He), neon (Ne), argon (Ar), krypton (Kr), xenon (Xe), and the radioactive radon (Rn).

Purified argon and nitrogen gases are most commonly used as inert gases due to their high natural abundance (78.3% N₂, 1% Ar in air) and low relative cost. The preferred is nitrogen (N₂) as the inert gas for diluting the elemental fluorine (F₂) in the fluorination gas to the desired but still high concentration, as defined herein.

Preferred is a fluorination gas, wherein the elemental fluorine (F₂) is diluted by nitrogen (N₂). An example composition of a fluorination gas, using nitrogen (N₂) as the inert gas, is as follows (here as purified composition (fluorine-nitrogen gas mixture) as filled in a steel gas cylinder):

| Molecular Formula: F₂ | Molecular Weight: 38 |
|---|---|
| Item | Index |
| F₂ content (volume fraction)/10⁻² | 20 |
| N₂ content (volume fraction)/10⁻² | 80 |
| O₂ content (volume fraction)/10⁻² | ≤0.08 |
| CF₄ content (volume fraction)/10⁻² | ≤0.03 |
| HF content (volume fraction)/10⁻² | ≤0.50 |

Properties: melting point: −218° C., boiling point: −187° C., relative densitiy (moisture = 1) 1.14 (−200° C.), soluble in water, relative density (air = 1) 1.70, saturated vapor pressure (kpa): 101.32 (−187° C.), critical pressure (MPA): 5.57.

Fluorination Reactions:

The direct fluorination reactions of the current invention can be performed in a solvent which is inert to fluorination under the reaction conditions. As stated before, in the process of the invention the inert solvent is selected from the group consisting of hydrogen fluoride (HF), anhydrous hydrogen fluoride (anhydrous HF), Olah's reagent (pyridine/HF), acetonitrile (CH₃CN), formic acid, and trifluoroacetic acid, fluorinated alkane pentafluorobutane (365mfc), fluorinated ether CF₃—CH₂—OCHF₂ (E245, perhalogenated ether CF₃—O—CF₂—CCl₃, octafluorotetrahydrofurane, perhalogenated compound CFCl₃, and perhalogenated compound CF₂Cl—CFCl₂ (113). All these inert solvents have good solubility for the materials, and inertness against elemental fluorine (F₂) and hydrogen fluoride (HF).

For example, the inert solvent may be anhydrous HF, Olah's reagent (pyridin/HF: allows easier preparation of starting material solution but is more difficult to remove out of product after reaction), acetonitrile (CH₃CN), formic acid, and trifluoroacetic acid or also perhalogenated compounds like CFCl₃ (to be used under higher pressures only), CF₂Cl—CCl₂F (113, has a preferred boiling point of 48° C.) and also perhalogenated ethers like e.g. CF₃—O—CF₂—CCl₃ (b.p. 87° C.), formic acid and trifluoroacetic acid.

In such a case, wherein the fluorination is carried out in a solvent, then the direct fluorination according to the invention is advantageously performed using slightly sub-molar amounts of the fluorination gas comprising highly concentrated F₂-gas. This is particularly the case when the starting compounds are solids, and therefore, a solvent (e.g., acetonitrile) is used.

Further, it has been discovered that despite the exothermic character of the direct fluorination reaction, e.g., within a given time period (e.g., less than 10 hours, or even less than 5 hours), the reaction of the invention can be performed as a larger scale reaction with high conversion rates, and without major impurities in the resulting fluorinated product. The fluorinated product can be produced in kilogram scale quantities and up to (metric) ton scale quantities (1 metric ton corresponds to 1,000 kg), respectively, e.g., the direct fluorination process of the invention can be performed in a large-scale and/or industrial production of the fluorinated compound, as defined herein before according to the invention. The terms “large-scale production” and/or “industrial production”, thus, each are meant to define a production scale in the range of starting at, for example, about 1 kilogram, and ranging up to about several (metric) tons (e.g., about 1, about 2, about 3, about 4, about 5, about 6, about 7, about 8, about 9, about 10, even up to about tens of (metric) tons). For example, The terms “large-scale production” and/or “industrial production”, thus, each are meant to define a production scale in the range, for example, each of at least: about 1 kilogram, about 2 kilograms, about 3 kilograms, about 4 kilograms, about 5 kilograms, about 6 kilograms, about 7 kilograms, about 8 kilograms, about 9 kilograms, about 10 kilograms, about 15 kilograms, about 20 kilograms, about 25 kilograms, about 30 kilograms, about 35 kilograms, about 40 kilograms, about 45 kilograms, about 50 kilograms, about 100 kilograms, about 150 kilograms, about 200 kilograms, about 250 kilograms, about 300 kilograms, about 350 kilograms, about 400 kilograms, about 450 kilograms, about 500 kilograms, about 600 kilograms, about 700 kilograms, about 800 kilograms, about 900 kilograms; or, the terms “large-scale production” and/or “industrial production”, thus, each are meant to define a production scale in the range, for example, each of at least: about 1 (metric) ton, about 2 (metric) ton, about 3 (metric) ton, about 4 (metric) ton, about 5 (metric) ton, about 6 (metric) ton, about 7 (metric) ton, about 8 (metric) ton, about 9 (metric) ton, about 10 (metric) tons.

Accordingly, it is preferred that the direct fluorination process of the invention is performed in a large-scale and/or industrial production of the fluorinated compound, as defined herein before according to the invention, e.g., at least in in kilogram scale quantities, but preferably, in “large-scale production” and/or “industrial production” scale as defined herein before.

The reaction is performed with an equimolar amount of highly concentrated F₂-gas, and optionally in a slight molar excess amount of about 0.01 to about 0.1 mol/h, but preferably in a slight sub-molar amount of about −0.01 to about 0.1 mol/h, more preferably in a slight sub-molar amount of about −0.02 to about −0.09 mol/h, even more preferably of about −0.03 to about −0.08 mol/h, most preferably of about −0.5 to about −0.07 mol/h, of highly concentrated F₂-gas.

Fluorination with Fluorination Gas Containing Elemental Fluorine in a High Concentration:

As briefly described, and defined in the claims and further detailed by the following description and examples herein, the invention is particularly directed to a use of a fluorination gas, wherein the elemental fluorine (F₂) is present in a high concentration the process for the manufacture of the fluorinated compound, as defined herein before, by direct fluorination employing a fluorination gas, wherein the elemental fluorine (F₂) is present in a high concentration. This particular aspect of the invention shall be further explained herein after.

As shown in the examples, the direct fluorination can be performed already with a fluorination gas, based on the total fluorination gas composition as 100% by volume, comprising at least 20% by volume of elemental fluorine (F₂) and up to about 80% by volume of an inert gas, preferably nitrogen (N₂), for example, the composition of a fluorination gas, using nitrogen (N₂) as the inert gas, as described above as purified composition fluorine-nitrogen gas mixture as filled in a steel gas cylinder.

The use of inert gas in larger ratios of inert gas to elemental fluorine has disadvantages in terms of process controllability of the fluorination reaction, for example, in terms of effective mixing of the elemental fluorine (F₂) with the liquid compound to be fluorinated, heat transfer control, e.g., poor heat exchange, and maintenance of desired reaction conditions in the micro-environments in the reaction mixture. These disadvantages equally apply in bed tower reactor (gas scrubber system) technology and in microbubble microreactor or comparable continuous flow technology. For example, in a coil reactor or microreactor, at high inert gas concentrations, e.g., low fluorine (F₂) concentrations, in addition to the poor heat exchange, there are also ineffective (reaction) zones with (inert) gas bubbles, which nullifies the advantages of using a coil reactor or a microreactor, and the same is observed in bed tower reactor (gas scrubber system) technology.

However, it was also found by the present invention that, based on the total fluorination gas composition as 100% by volume, increasing the concentration of elemental fluorine (F₂) in the fluorination gas to a higher concentration of greater than 20% by volume, e.g., preferably of greater than 25% by volume, more preferably of greater than 30% by volume or 40% by volume, and most preferably of greater than 50% by volume, while on the other hand decreasing the concentration of the inert gas, e.g., of the inert gas nitrogen (N₂), to a corresponding lower concentration of less than 80% by volume, e.g., preferably of less than 75% by volume, more preferably of less than 70% by volume or 60% by volume, and most preferably of less than 50% by volume, for an industrial process gradually increasing conversion rates of essentially above about 30 to 45%, e.g. conversion rates of more than 50% by volume, preferably of more than 60% by volume, or more than 70% by volume, or more than 70% by volume, even more preferably of more than 80% by volume, and most preferably of more than 90% by volume, can be achieved.

Without wishing to be bound to a theory, it is estimated that the inert gas used to dilute the reactivity of the strongly oxidant elemental fluorine (F₂), which is required for safety reasons when handling and transporting elemental fluorine (F₂) as described in the background above (e.g., in Europe mixtures of 95% by volume N2 (inert gas) with only 5% by volume F₂-gas, or in Asia, e.g., at least 80% by volume N2 (inert gas) with only up to 20% by volume F₂-gas) is jeopardizing the fluorination reaction, despite the fact that the elemental fluorine (F₂) contained in such a diluted fluorination gas still is strong oxidant.

Surprisingly, by the present invention it was found, that direct fluorination of compounds with even higher conversion rates than those obtained with the said conventional diluted fluorination gases can be achieved, if the elemental fluorine (F₂) is undiluted by inert gas, or elemental fluorine (F₂) is diluted by inert gas only to a concentration of greater than 50% by volume elemental fluorine (F₂) in the fluorination gas, based on the total fluorination gas composition as 100% by volume.

Therefore, it is particularly preferred by the present invention to provide a fluorination process for the manufacture of the fluorinated compound by direct fluorination using fluorine gas (F₂), as it comes directly out of a F₂-electrolysis reactor (fluorine cell).

A representative composition of fluorine gas produced by a fluorine cell is 97% F₂, up to 3% CF₄ (formed from damage of the electrodes), traces of HF, NO₂, OF₂, COF₂, each % by volume and based on the total volume of the fluorine containing gas as 100% by volume.

Purification of the fluorination gas as it is derived from a F₂-electrolysis reactor (fluorine cell), if desired, optionally is possible, to remove a part or all by-products and traces formed in the F₂-electrolysis reactor (fluorine cell), prior to its use as fluorination gas in the process of the present invention. However, in the process of the present invention such a partial or complete purification is not required, and the fluorination gas can be directly used, as it comes directly out of a F₂-electrolysis reactor (fluorine cell).

When employing a fluorination gas derived from a F₂-electrolysis reactor (fluorine cell), purified or unpurified, it may, if desired, optionally be diluted to some extent by an inert gas, preferably by nitrogen (N₂).

Hence, such a fluorination gas, purified or unpurified, as it is derived from a F₂-electrolysis reactor (fluorine cell), if desired, may optionally be diluted by up to about 45% by volume of inert gas, but preferably the fluorination gas is not diluted by inert gas to a concentration of elemental fluorine (F₂) in the fluorination gas of less 80% by volume, preferably of less than 85% by volume, more preferably of less than 90% by volume, based on the total fluorination gas composition as 100% by volume.

The difference of the sum of the elemental fluorine (F₂) and any inert gas in the fluorination gas to 100% by volume, if any difference, may be constituted by by-products (e.g., CF₄) and traces of HF, NO₂, OF₂, COF₂, formed from damage of the electrodes of the F₂-electrolysis reactor (fluorine cell). This applies generally to the % by volume values given herein above and herein below, if fluorine gas (F₂), as it comes directly out of a F₂-electrolysis reactor (fluorine cell) is used as the fluorination gas in the present invention.

Accordingly, in a preferred process of the invention the direct fluorination is carried out with a fluorination gas comprising about 80% by volume to 97±1% of elemental fluorine (F₂) and about 0% to 17±1% of inert gas, preferably of nitrogen (N₂), based on the total fluorination gas composition as 100% by volume.

In a further preferred process of the invention the direct fluorination is carried out with a fluorination gas comprising about 85% by volume to 97±1% of elemental fluorine (F₂) and about 0% to 12±1% of inert gas, preferably of nitrogen (N₂), based on the total fluorination gas composition as 100% by volume.

In a furthermore preferred process of the invention the direct fluorination is carried out with a fluorination gas comprising about 87% by volume to 97±1% of elemental fluorine (F₂) and about 0% to 10±1% of inert gas, preferably of nitrogen (N₂), based on the total fluorination gas composition as 100% by volume.

In another preferred process of the invention the direct fluorination is carried out with a fluorination gas comprising about 90% by volume to 97±1% of elemental fluorine (F₂) and about 0% to 7±1% of inert gas, preferably of nitrogen (N₂), based on the total fluorination gas composition as 100% by volume.

In still another preferred process of the invention the direct fluorination is carried out with a fluorination gas comprising about 95% by volume to 97±1% of elemental fluorine (F₂) and about 0% to 2±1% of inert gas, preferably of nitrogen (N₂), based on the total fluorination gas composition as 100% by volume.

It goes without saying that a person skilled in the art understands that within any of the given ranges any intermediate values and intermediate ranges can be selected, too.

Batch Process:

The invention also may pertain to a process for the manufacture of the fluorinated compound, wherein the process is a batchwise process, preferably wherein the batchwise process is carried out in a column reactor. Although, in the following reactor setting the process is described as a batch process, as preferred, for example, in case of high product concentrations, optionally the process can be performed in the said reactor setting also as a continuous process. In case of a continuous process in the said reactor setting, then, it goes without saying, the additional inlet(s) and outlet(s) are foreseen, for feeding the starting compound and withdrawing the product compound, respectively.

If the invention pertains to a batchwise process, preferably wherein the batchwise process is carried out in a column reactor, the process for the manufacture of the fluorinated compound, most preferably the reaction is carried out in a (closed) column reactor (system), wherein the liquid medium of a) comprising or consisting of the starting compound is circulated in a loop, while the fluorination gas of b) comprising or consisting of elemental fluorine (F₂) in a high concentration is fed into the column reactor of c) and in step d) is passed through the liquid medium to react with the starting compound; preferably wherein the loop is operated with a circulation velocity of from 1,500 l/h to 5,000 l/h, more preferably of from 3,500 l/h to 4,500 l/h.

If the invention pertains to a batchwise process, the process for the manufacture of the fluorinated compound, as defined according to the invention, can be carried out such that the liquid medium of a) comprising or consisting of the starting compound is circulated in the column reactor in a turbulent stream or in laminar stream, preferably in a turbulent stream.

In general, the fluorination gas containing the elemental fluorine (F₂) is fed into the loop in accordance with the required stoichiometry for the targeted fluorinated product and fluorination degree, and adapted to the reaction rate.

For example, the said process for the manufacture of a fluorinated compound, as defined according to the invention, may be performed, e.g., batchwise, wherein the column reactor is equipped with at least one of the following: at least one cooler (system), at least one liquid reservoir for the liquid medium of a) comprising or consisting of a starting compound, a pump (for pumping/circulating the liquid medium), one or more (nozzle) jets, preferably placed at the top of the column reactor, for spraying the circulating medium into the column reactor, or alternatively (instead of the one or more (nozzle) jets) a perforated metal sheet placed at the top of the column reactor, for circulating the liquid medium of (i) into the column reactor, used together with a high-efficiency pump, one or more feeding inlets for introducing the fluorination gas of b) comprising or consisting of elemental fluorine (F₂) in a high concentration, optionally one or more sieves, preferably two sieves, preferably the one or more sieves placed at the bottom of the column reactor, at least one gas outlet equipped with a pressure valve.

A so-called perforated metal sheet in particular can be used if the pump performance allows for it, e.g., in case of a high-efficiency pump. In case, the use of a so-called perforated metal sheet can be advantageous, for example, is there is a potential risk of clogging (nozzle) jets.

In one embodiment, the process for the manufacture of the fluorinated compound, as defined according to the invention, can be performed in a column reactor is a packed bed tower reactor, preferably a packed bed tower reactor which is packed with fillers resistant to elemental fluorine (F₂) and hydrogen fluoride (HF), e.g. with Raschig fillers and/or metal fillers, more preferably wherein the packed bed tower reactor is a gas loop (scrubber) system (tower) which is packed with fillers resistant to elemental fluorine (F₂) and hydrogen fluoride (HF), e.g. Raschig fillers and/or metal fillers.

In a further embodiment, the process for the manufacture of the fluorinated compound, as defined according to the invention, the reaction is carried out with a counter-current flow of the circulating liquid medium of a) comprising or consisting of the starting compound and of the fluorination gas of b) fed into the column reactor and which fluorination gas of b) is comprising or consisting of elemental fluorine (F₂) in a high concentration.

The pressure valve functions to keep the pressure, as required in the reaction, and to release any effluent gas, e.g. inert carrier gas contained in the fluorination gas, if applicable together with any hydrogen fluoride (HF) released for the reaction.

The said process for the manufacture of the fluorinated compound, as defined according to the invention, may be performed, e.g., batchwise, such that in the said process for the manufacture of the fluorinated compound the column reactor is a packed bed tower reactor, preferably a packed bed tower reactor which is packed with metal fillers.

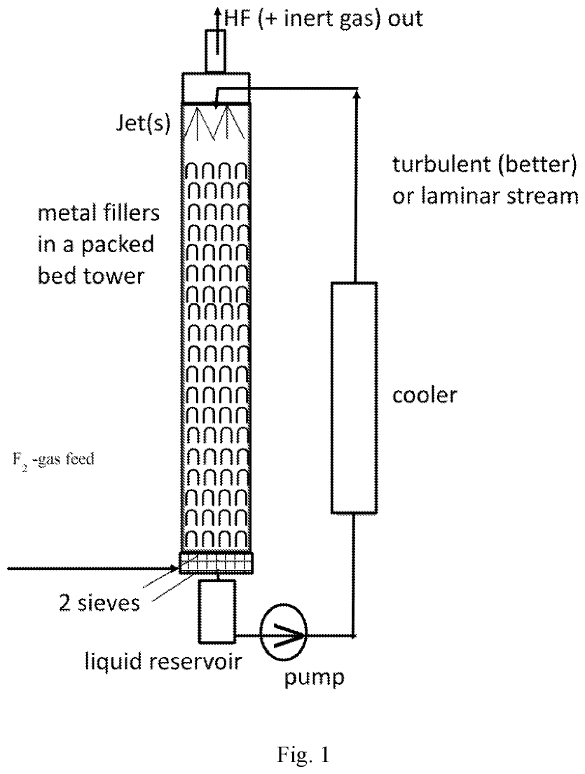

In FIG. 1, batch fluorination with F₂-gas in a counter-current system (the starting material reservoir is containing the liquid starting material or optionally the starting material in an inert solvent). If an F₂ is used which is diluted with some inert gas (90% or 80% N₂ or other inert gases like He or Ar) the pressure during the fluorination is kept at 20 bar by a pressure valve. The inert gas together with (only) some HF leaves as purge gas during reaction. For concentrated F₂-gas prepared “in situ” in a state of the art electrolysis cell or 2-onsite generators like offered by Linde (https://www.linde-gas.com/en/products_and_supply/electronic_gases_and_chemicals/on_site_gas_g eneration/generation_f_on_site_fluorine_generation/index.html). The packed-bed reactor, for example, is equipped with (resistant) metal fillers, and can also be equipped with HDPTFE-fillers (Raschig). Typical filling materials for a packed-bed reactor have a diameter of not smaller than about 1 cm (e.g., not smaller than about 1±0.05 cm).

The packed tower according to FIG. 1 can have a diameter of 100 or 200 mm (depending on the circulating flow rate and scale) made out of high grade stainless steel (1.4571) or Hastelloy (preferred Hastelloy C4) and a length of 3 meters for the 100 mm and a length of 6 meters for the 200 mm diameter tower (latter if higher capacities are needed). The tower made is filled either with E-TFE- or HDPTFE-fillings, or metal fillings each of 10 mm diameter as available from Raschig (http://www.raschig.de/Fllkrper). The type of fillings is quite flexible, Raschig Pall-Rings made out of Hastelloy were used in the trials disclosed hereunder, also E-TFE-fillings showed same performance, both not causing too much pressure reduction (pressure loss) while feeding F₂-gas in counter-current manner. Plastics (e.g. HD-PTFE) as construction material for the tower are also suitable but for lower pressures only. If plastics are used, measures have to be taken to avoid electrostatic charges.

In the process for the manufacture of the fluorinated compound, as defined according to any of the embodiments of the invention, the reaction may be carried out with a counter-current flow of circulating liquid medium of a) comprising or consisting of the starting compound and the fluorination gas of b) fed into the column reactor and comprising or consisting of elemental fluorine (F₂) in a high concentration.

Microreactor Process:

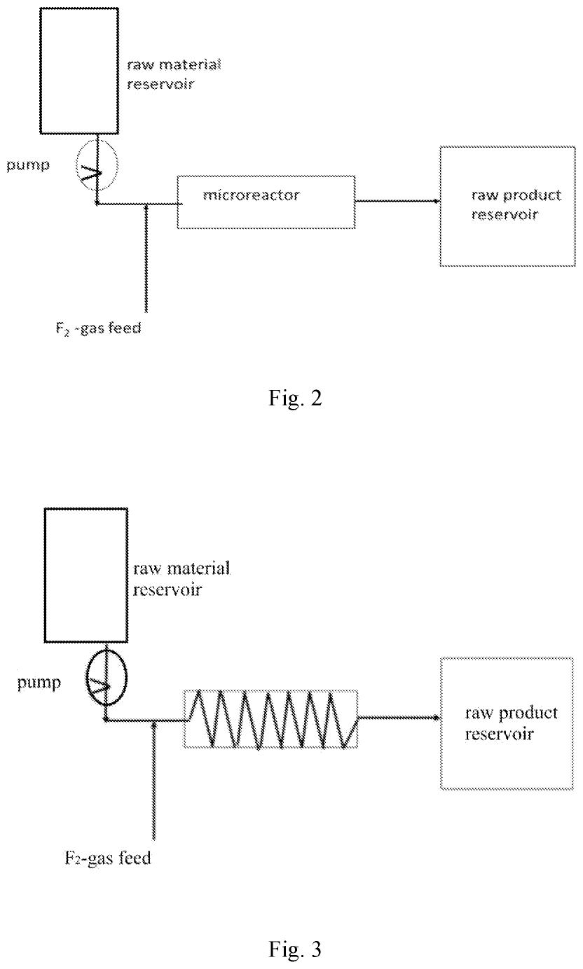

The invention also may pertain to a process for the manufacture of the fluorinated compound, as defined according to any of the embodiments of the invention, wherein the process is a continuous process, preferably wherein the continuous process is carried out in a microreactor. See FIG. 2.

In general, the fluorination gas containing the elemental fluorine (F₂) is fed into the microreactor in accordance with the required stoichiometry (sometimes with a slight excess) for the targeted fluorinated product and fluorination degree, and adapted to the reaction rate.

The invention may employ more than a single microreactor, i.e., the invention may employ two, three, four, five or more microreactors, for either extending the capacity or residence time, for example, to up to ten microreactors in parallel or four microreactors in series. If more than a single microreactor is employed, then the plurality of microreactors can be arranged either sequentially or in parallel, and if three or more microreactors are employed, these may be arranged sequentially, in parallel or both.

The invention is also very advantageous, in one embodiment wherein the direct fluorination of the invention optionally is performed in a continuous flow reactor system, or preferably in a microreactor system.

In an preferred embodiment the invention relates to a process for the manufacture of a fluorinated compound according to the invention, wherein the reaction is carried out in at least one step as a continuous processes, wherein the continuous process is performed in at least one continuous flow reactor with upper lateral dimensions of about ≤5 mm, or of about ≤4 mm, as further defined already above in more detail.

In another preferred embodiment the invention relates to such a process of preparing a compound according to the invention, wherein at least one of the said continuous flow reactors, preferably at least one of the microreactors, independently is a SiC-continuous flow reactor, preferably independently is a SiC-microreactor.

The Continuous Flow Reactors and Microreactors:

In addition to the above, according to one aspect of the invention, also a plant engineering invention is provided, as used in the process invention and described herein, pertaining to the optional, and in some embodiments of the process invention, the process even preferred implementation in microreactors.

As to the term “microreactor”: A “microreactor” or “microstructured reactor” or “microchannel reactor”, in one embodiment of the invention, is a device in which chemical reactions take place in a confinement with typical lateral dimensions of about ≤1 mm; an example of a typical form of such confinement are microchannels. Generally, in the context of the invention, the term “microreactor”: A “microreactor” or “microstructured reactor” or “microchannel reactor”, denotes a device in which chemical reactions take place in a confinement with typical lateral dimensions of about ≤5 mm.

Microreactors are studied in the field of micro process engineering, together with other devices (such as micro heat exchangers) in which physical processes occur. The microreactor is usually a continuous flow reactor (contrast with/to a batch reactor). Microreactors offer many advantages over conventional scale reactors, including vast improvements in energy efficiency, reaction speed and yield, safety, reliability, scalability, on-site/on-demand production, and a much finer degree of process control.

Microreactors are used in “flow chemistry” to perform chemical reactions.

In flow chemistry, wherein often microreactors are used, a chemical reaction is run in a continuously flowing stream rather than in batch production. Batch production is a technique used in manufacturing, in which the object in question is created stage by stage over a series of workstations, and different batches of products are made. Together with job production (one-off production) and mass production (flow production or continuous production) it is one of the three main production methods. In contrast, in flow chemistry the chemical reaction is run in a continuously flowing stream, wherein pumps move fluid into a tube, and where tubes join one another, the fluids contact one another. If these fluids are reactive, a reaction takes place. Flow chemistry is a well-established technique for use at a large scale when manufacturing large quantities of a given material. However, the term has only been coined recently for its application on a laboratory scale.

Continuous flow reactors, e.g. such as used as microreactor, are typically tube like and manufactured from non-reactive materials, such known in the prior art and depending on the specific purpose and nature of possibly aggressive agents and/or reactants. Mixing methods include diffusion alone, e.g. if the diameter of the reactor is narrow, e.g. <1 mm, such as in microreactors, and static mixers. Continuous flow reactors allow good control over reaction conditions including heat transfer, time and mixing. The residence time of the reagents in the reactor, i.e. the amount of time that the reaction is heated or cooled, is calculated from the volume of the reactor and the flow rate through it: Residence time=Reactor Volume/Flow Rate. Therefore, to achieve a longer residence time, reagents can be pumped more slowly, just a larger volume reactor can be used and/or even several microreactors can be placed in series, optionally just having some cylinders in between for increasing residence time if necessary for completion of reaction steps. In this later case, cyclones after each microreactor help to let formed HCl to escape and to positively influence the reaction performance. Production rates can vary from milliliters per minute to liters per hour.

Some examples of flow reactors are spinning disk reactors (Colin Ramshaw); spinning tube reactors; multi-cell flow reactors; oscillatory flow reactors; microreactors; hex reactors; and aspirator reactors. In an aspirator reactor a pump propels one reagent, which causes a reactant to be sucked in. Also to be mentioned are plug flow reactors and tubular flow reactors.

In the present invention, in one embodiment it is particularly preferred to employ a microreactor.

In the use and processes according to the invention in a preferred embodiment the invention is using a microreactor. But it is to be noted in a more general embodiment of the invention, apart from the said preferred embodiment of the invention that is using a microreactor, any other, e.g. preferentially pipe-like, continuous flow reactor with upper lateral dimensions of up to about 1 cm, and as defined herein, can be employed. Thus, such a continuous flow reactor preferably with upper lateral dimensions of up to about ≤5 mm, or of about ≤4 mm, refers to a preferred embodiment of the invention, e.g. preferably to a microreactor. Continuously operated series of STRs is another option, but less preferred than using a microreactor.

In the before said embodiments of the invention, the minimal lateral dimensions of the, e.g. preferentially pipe-like, continuous flow reactor can be about >5 mm; but is usually not exceeding about 1 cm. Thus, the lateral dimensions of the, e.g. preferentially pipe-like, continuous flow reactor can be in the range of from about >5 mm up to about 1 cm, and can be of any value therein between. For example, the lateral dimensions of the, e.g. preferentially pipe-like, continuous flow reactor can be about 5.1 mm, about 5.5 mm, about 6 mm, about 6.5 mm, about 7 mm, about 7.5 mm, about 8 mm, about 8.5 mm, about 9 mm, about 9.5 mm, and about 10 mm, or can be can be of any value intermediate between the said values.

In the before said embodiments of the invention using a microreactor preferentially the minimal lateral dimensions of the microreactor can be at least about 0.25 mm, and preferably at least about 0.5 mm; but the maximum lateral dimensions of the microreactor does not exceed about ≤5 mm. Thus, the lateral dimensions of the, e.g. preferential microreactor can be in the range of from about 0.25 mm up to about ≤5 mm, and preferably from about 0.5 mm up to about ≤5 mm, and can be of any value therein between. For example, the lateral dimensions of the preferential microreactor can be about 0.25 mm, about 0.3 mm, about 0.35 mm, about 0.4 mm, about 0.45 mm, and about 5 mm, or can be can be of any value intermediate between the said values.

As stated here before in the embodiments of the invention in its broadest meaning is employing, preferentially pipe-like, continuous flow reactor with upper lateral dimensions of up to about 1 cm. Such continuous flow reactor, for example is a plug flow reactor (PFR).

The plug flow reactor (PFR), sometimes called continuous tubular reactor, CTR, or piston flow reactors, is a reactor used to perform and describe chemical reactions in continuous, flowing systems of cylindrical geometry. The PFR reactor model is used to predict the behavior of chemical reactors of such design, so that key reactor variables, such as the dimensions of the reactor, can be estimated.

Fluid going through a PFR may be modeled as flowing through the reactor as a series of infinitely thin coherent “plugs”, each with a uniform composition, traveling in the axial direction of the reactor, with each plug having a different composition from the ones before and after it. The key assumption is that as a plug flows through a PFR, the fluid is perfectly mixed in the radial direction (i.e. in the lateral direction) but not in the axial direction (forwards or backwards).

Accordingly, the terms used herein to define the reactor type used in the context of the invention such like “continuous flow reactor”, “plug flow reactor”, “tubular reactor”, “continuous flow reactor system”, “plug flow reactor system”, “tubular reactor system”, “continuous flow system”, “plug flow system”, “tubular system” are synonymous to each other and interchangeably by each other.

The reactor or system may be arranged as a multitude of tubes, which may be, for example, linear, looped, meandering, circled, coiled, or combinations thereof. If coiled, for example, then the reactor or system is also called “coiled reactor” (“coil reactor”) or “coiled system”, for example, a coiled or coil reactor as shown in FIG. 3. If looped, for example, then the reactor or system is also called “loop reactor” or “loop system”, for example, a loop reactor as shown in FIG. 4. The column reactor as shown in FIG. 1, is also regarded as “loop reactor” or “loop system”, e.g., as a counter-current (loop) system (“inverse gas scrubber system”).

In the radial direction, i.e. in the lateral direction, such reactor or system may have an inner diameter or an inner cross-section dimension (i.e. radial dimension or lateral dimension, respectively) of up to about 1 cm. Thus, in an embodiment the lateral dimension of the reactor or system may be in the range of from about 0.25 mm up to about 1 cm, preferably of from about 0.5 mm up to about 1 cm, and more preferably of from about 1 mm up to about 1 cm.

In further embodiments the lateral dimension of the reactor or system may be in the range of from about >5 mm to about 1 cm, or of from about 5.1 mm to about 1 cm.

If the lateral dimension at maximum of up to about ≤5 mm, or of up to about ≤4 mm, then the reactor is called “microreactor”. Thus, in still further microreactor embodiments the lateral dimension of the reactor or system may be in the range of from about 0.25 mm up to about ≤5 mm, preferably of from about 0.5 mm up to about ≤5 mm, and more preferably of from about 1 mm up to about ≤5 mm; or the lateral dimension of the reactor or system may be in the range of from about 0.25 mm up to about ≤4 mm, preferably of from about 0.5 mm up to about ≤4 mm, and more preferably of from about 1 mm up to about ≤4 mm.

In case reactants are solid or oily inert solvents may be used. Thus, if solid and/or oily starting materials shall be used, then the said solid and/or oily materials are dissolved in an inert solvent. Any suitable inert solvent, as identified above, can be used in the process. For example, acetonitrile, formic acid, trifluoroacetic acid, or fully or partially fluorinated alkanes like pentafluorobutane (365mfc), linear or cyclic partially or fully fluorinated ethers like CF₃—CH₂—OCHF₂ (E245) or octafluorotetrahydrofurane, and linked to inertness against elemental fluorine (F₂), fully fluorinated (or at least fully halogenated) solvents, for example, such as CFCl₃, CF₂Cl—CFCl₂, are preferred.

In an alternative embodiment of the invention, it is also optionally desired to employ another continuous flow reactor than a microreactor, preferably if, for example, the (halogenation promoting, e.g. the halogenation or preferably the halogenation) catalyst composition used in the halogenation or fluorination tends to get viscous during reaction or is viscous already as a said catalyst as such. In such case, a continuous flow reactor, i.e. a device in which chemical reactions take place in a confinement with lower lateral dimensions of greater than that indicated above for a microreactor, i.e. of greater than about 1 mm, but wherein the upper lateral dimensions are about ≤4 mm. Accordingly, in this alternative embodiment of the invention, employing a continuous flow reactor, the term “continuous flow reactor” preferably denotes a device in which chemical reactions take place in a confinement with typical lateral dimensions of from about ≥1 mm up to about ≤4 mm. In such an embodiment of the invention it is particularly preferred to employ as a continuous flow reactor a plug flow reactor and/or a tubular flow reactor, with the said lateral dimensions. Also, in such an embodiment of the invention, as compared to the embodiment employing a microreactor, it is particularly preferred to employ higher flow rates in the continuous flow reactor, preferably in the plug flow reactor and/or a tubular flow reactor, with the said lateral dimensions. For example, such higher flow rates, are up to about 2 times higher, up to about 3 times higher, up to about 4 times higher, up to about 5 times higher, up to about 6 times higher, up to about 7 times higher, or any intermediate flow rate of from about ≥1 up to about ≤7 times higher, of from about ≥1 up to about ≤6 times higher, of from about ≥1 up to about ≤5 times higher, of from about ≥1 up to about ≤4 times higher, of from about ≥1 up to about ≤3 times higher, or of from about ≥1 up to about ≤2 times higher, each as compared to the typical flow rates indicated herein for a microreactor. Preferably, the said continuous flow reactor, more preferably the plug flow reactor and/or a tubular flow reactor, employed in this embodiment of the invention is configured with the construction materials as defined herein for the microreactors. For example, such construction materials are silicon carbide (SiC) and/or are alloys such as a highly corrosion resistant nickel-chromium-molybdenum-tungsten alloy, e.g. Hastelloy®, as described herein for the microreactors.

A very particular advantage of the present invention employing a microreactor, or a continuous flow reactor with the before said lateral dimensions, the number of separating steps can be reduced and simplified, and may be devoid of time and energy consuming, e.g. intermediate, distillation steps. Especially, it is a particular advantage of the present invention employing a microreactor, or a continuous flow reactor with the before said lateral dimensions, that for separating simply phase separation methods can be employed, and the non-consumed reaction components may be recycled into the process, or otherwise be used as a product itself, as applicable or desired.

In addition to the preferred embodiments of the present invention using a microreactor according to the invention, in addition or alternatively to using a microreactor, it is also possible to employ a plug flow reactor or a tubular flow reactor, respectively.

Plug flow reactor or tubular flow reactor, respectively, and their operation conditions, are well known to those skilled in the field.

Although the use of a continuous flow reactor with upper lateral dimensions of about ≤5 mm, or of about ≤4 mm, respectively, and in particular of a microreactor, is particularly preferred in the present invention, depending on the circumstances, it could be imagined that somebody dispenses with an microreactor, then of course with yield losses and higher residence time, higher temperature, and instead takes a plug flow reactor or turbulent flow reactor, respectively. However, this could have a potential advantage, taking note of the mentioned possibly disadvantageous yield losses, namely the advantage that the probability of possible blockages (tar particle formation by non-ideal driving style) could be reduced because the diameters of the tubes or channels of a plug flow reactor are greater than those of a microreactor.

The possibly allegeable disadvantage of this variant using a plug flow reactor or a tubular flow reactor, however, may also be seen only as subjective point of view, but on the other hand under certain process constraints in a region or at a production facility may still be appropriate, and loss of yields be considered of less importance or even being acceptable in view of other advantages or avoidance of constraints.

In the following, the invention is more particularly described in the context of using a microreactor. Preferentially, a microreactor used according to the invention is a ceramic continuous flow reactor, more preferably an SiC (silicon carbide) continuous flow reactor, and can be used for material production at a multi-to scale. Within integrated heat exchangers and SiC materials of construction, it gives optimal control of challenging flow chemistry application. The compact, modular construction of the flow production reactor enables, advantageously for: long term flexibility towards different process types; access to a range of production volumes (5 to 400 l/h); intensified chemical production where space is limited; unrivalled chemical compatibility and thermal control.

Ceramic (SiC) microreactors, are e.g. advantageously diffusion bonded 3M SiC reactors, especially braze and metal free, provide for excellent heat and mass transfer, superior chemical compatibility, of FDA certified materials of construction, or of other drug regulatory authority (e.g. EMA) certified materials of construction. Silicon carbide (SiC), also known as carborundum, is a containing silicon and carbon, and is well known to those skilled in the art. For example, synthetic SiC powder is been mass-produced and processed for many technical applications.

For example, in the embodiments of the invention the objects are achieved by a method in which at least one reaction step takes place in a microreactor. Particularly, in preferred embodiments of the invention the objects are achieved by a method in which at least one reaction step takes place in a microreactor that is comprising or is made of SiC (“SiC-microreactor”), or in a microreactor that is comprising or is made of an alloy, e.g. such as Hastelloy C, as it is each defined herein after in more detail.

Thus, without being limited to, for example, in an embodiment of the invention the microreactor suitable for, preferably for industrial, production an “SiC-microreactor” that is comprising or is made of SiC (silicon carbide; e.g. SiC as offered by Dow Corning as Type G1SiC or by Chemtrix MR555 Plantrix), e.g. providing a production capacity of from about 5 up to about 400 kg per hour; or without being limited to, for example, in another embodiment of the invention the microreactor suitable for industrial production is comprising or is made of Hastelloy C, as offered by Ehrfeld. Such microreactors are particularly suitable for the, preferably industrial, production of fluorinated products according to the invention.

In order to meet both the mechanical and chemical demands placed on production scale flow reactors, Plantrix modules are fabricated from 3M™ SiC (Grade C). Produced using the patented 3M (EP 1 637 271 B1 and foreign patents) diffusion bonding technology, the resulting monolithic reactors are hermetically sealed and are free from welding lines/joints and brazing agents. More technical information on the Chemtrix MR555 Plantrix can be found in the brochure “CHEMTRIX—Scalable Flow Chemistry—Technical Information Plantrix® MR555 Series, published by Chemtrix BV in 2017, which technical information is incorporated herein by reference in its entirety.

Apart from the before said example, in other embodiments of the invention, in general SiC from other manufactures, and as known to the skilled person, of course can be employed in the present invention.

Accordingly, in the present invention as microreactor also the Protrix® of by Chemtrix can be used. Protrix® is a modular, continuous flow reactor fabricated from 3M® silicon carbide, offering superior chemical resistance and heat transfer. In order to meet both the mechanical and chemical demands placed on flow reactors, Protrix® modules are fabricated from 3M® SiC (Grade C). Produced using the patented 3M (EP 1 637 271 B1 and foreign patents) diffusion bonding technology, the resulting monolithic reactors are hermetically sealed and are free from welding lines/joints and brazing agents. This fabrication technique is a production method that gives solid SiC reactors (thermal expansion coefficient=4.1×10⁻⁶K⁻¹).

Designed for flow rates ranging from 0.2 to 20 ml/min and pressures up to 25 bar, Protrix® allows the user to develop continuous flow processes at the lab-scale, later transitioning to Plantrix® MR555 (×340 scale factor) for material production. The Protrix® reactor is a unique flow reactor with the following advantages: diffusion bonded 3M® SiC modules with integrated heat exchangers that offer unrivaled thermal control and superior chemical resistance; safe employment of extreme reaction conditions on a g scale in a standard fumehood; efficient, flexible production in terms of number of reagent inputs, capacity or reaction time. The general specifications for the Protrix® flow reactors are summarised as follows; possible reaction types are, e.g. A+B→P1+Q (or C)→P, wherein the terms “A”, “B” and “C” represent educts, “P” and “P1” products, and “Q” quencher; throughput (ml/min) of from about 0.2 up to about 20; channel dimensions (mm) of 1×1 (pre-heat and mixer zone), 1.4×1.4 (residence channel); reagent feeds of 1 to 3; module dimensions (width×height) (mm) of 110×260; frame dimensions (width×height×length) (mm) approximately 400×300×250; number of modules/frame is one (minimum) up to four (max.). More technical information on the Chemtrix Protrix® reactor can be found in the brochure “CHEMTRIX—Scalable Flow Chemistry—Technical Information Protrix®, published by Chemtrix BV in 2017, which technical information is incorporated herein by reference in its entirety.

The Dow Corning as Type G1SiC microreactor, which is scalable for industrial production, and as well suitable for process development and small production can be characterized in terms of dimensions as follows: typical reactor size (length×width×height) of 88 cm×38 cm×72 cm; typical fluidic module size of 188 mm×162 mm. The features of the Dow Corning as Type G1SiC microreactor can be summarized as follows: outstanding mixing and heat exchange: patented HEART design; small internal volume; high residence time; highly flexible and multipurpose; high chemical durability which makes it suitable for high pH compounds and especially hydrofluoric acid; hybrid glass/SiC solution for construction material; seamless scale-up with other advanced-flow reactors. Typical specifications of the Dow Corning as Type G1SiC microreactor are as follows: flow rate of from about 30 ml/min up to about 200 ml/min; operating temperature in the range of from about −60° C. up to about 200° C., operating pressure up to about 18 barg (“barg” is a unit of gauge pressure, i.e. pressure in bars above ambient or atmospheric pressure); materials used are silicon carbide, PFA (perfluoroalkoxy alkanes), perfluoroelastomer; fluidic module of 10 ml internal volume; options: regulatory authority certifications, e.g. FDA or EMA, respectively. The reactor configuration of Dow Corning as Type G1SiC microreactor is characterized as multipurpose and configuration can be customized. Injection points may be added anywhere on the said reactor.

Hastelloy® C is an alloy represented by the formula NiCr21Mo14W, alternatively also known as “alloy 22” or “Hastelloy® C-22. The said alloy is well known as a highly corrosion resistant nickel-chromium-molybdenum-tungsten alloy and has excellent resistance to oxidizing reducing and mixed acids. The said alloy is used in flue gas desulphurization plants, in the chemical industry, environmental protection systems, waste incineration plants, sewage plants. Apart from the before said example, in other embodiments of the invention, in general nickel-chromium-molybdenum-tungsten alloy from other manufactures, and as known to the skilled person, of course can be employed in the present invention. A typical chemical composition (all in weight-%) of such nickel-chromium-molybdenum-tungsten alloy is, each percentage based on the total alloy composition as 100%: Ni (nickel) as the main component (balance) of at least about 51.0%, e.g. in a range of from about 51.0% to about 63.0%; Cr (chromium) in a range of from about 20.0 to about 22.5%, Mo (molybdenum) in a range of from about 12.5 to about 14.5%, W (tungsten or wolfram, respectively) in a range of from about 2.5 to about 3.5%; and Fe (iron) in an amount of up to about 6.0%, e.g. in a range of from about 1.0% to about 6.0%, preferably in a range of from about 1.5% to about 6.0%, more preferably in a range of from about 2.0% to about 6.0%. Optionally, the percentage based on the total alloy composition as 100%, Co (cobalt) can be present in the alloy in an amount of up to about 2.5%, e.g. in a range of from about 0.1% to about 2.5%. Optionally, the percentage based on the total alloy composition as 100%, V (vanadium) can be present in the alloy in an amount of up to about 0.35%, e.g. in a range of from about 0.1% to about 0.35%. Also, the percentage based on the total alloy composition as 100%, optionally low amounts (i.e. ≤0.1%) of other element traces, e.g. independently of C (carbon), Si (silicon), Mn (manganese), P (phosphor), and/or S (sulfur). In such case of low amounts (i.e. ≤0.1%) of other elements, the said elements e.g. of C (carbon), Si (silicon), Mn (manganese), P (phosphor), and/or S (sulfur), the percentage based on the total alloy composition as 100%, each independently can be present in an amount of up to about 0.1%, e.g. each independently in a range of from about 0.01 to about 0.1%, preferably each independently in an amount of up to about 0.08%, e.g. each independently in a range of from about 0.01 to about 0.08%. For example, said elements e.g. of C (carbon), Si (silicon), Mn (manganese), P (phosphor), and/or S (sulfur), the percentage based on the total alloy composition as 100%, each independently can be present in an amount of, each value as an about value: C≤0.01%, Si≤0.08%, Mn≤0.05%, P≤0.015%, S≤0.02%. Normally, no traceable amounts of any of the following elements are found in the alloy compositions indicated above: Nb (niobium), Ti (titanium), Al (aluminum), Cu (copper), N (nitrogen), and Ce (cerium).

Hastelloy® C-276 alloy was the first wrought, nickel-chromium-molybdenum material to alleviate concerns over welding (by virtue of extremely low carbon and silicon contents). As such, it was widely accepted in the chemical process and associated industries, and now has a 50-year-old track record of proven performance in a vast number of corrosive chemicals. Like other nickel alloys, it is ductile, easy to form and weld, and possesses exceptional resistance to stress corrosion cracking in chloride-bearing solutions (a form of degradation to which the austenitic stainless steels are prone). With its high chromium and molybdenum contents, it is able to withstand both oxidizing and non-oxidizing acids, and exhibits outstanding resistance to pitting and crevice attack in the presence of chlorides and other halides. The nominal composition in weight-% is, based on the total composition as 100%: Ni (nickel) 57% (balance); Co (cobalt) 2.5% (max.); Cr (chromium) 16%; Mo (molybdenum) 16%; Fe (iron) 5%; W (tungsten or wolfram, respectively) 4%; further components in lower amounts can be Mn (manganese) up to 1% (max.); V (vanadium) up to 0.35% (max.); Si (silicon) up to 0.08% (max.); C (carbon) 0.01 (max.); Cu (copper) up to 0.5% (max.).

In another embodiments of the invention, without being limited to, for example, the microreactor suitable for the said production, preferably for the said industrial production, is an SiC-microreactor that is comprising or is made only of SiC as the construction material (silicon carbide; e.g. SiC as offered by Dow Corning as Type G1SiC or by Chemtrix MR555 Plantrix), e.g. providing a production capacity of from about 5 up to about 400 kg per hour.

It is of course possible according to the invention to use one or more microreactors, preferably one or more SiC-microreactors, in the production, preferably in the industrial production, of the fluorinated products according to the invention. If more than one microreactor, preferably more than one SiC-microreactors, are used in the production, preferably in the industrial production, of the fluorinated products according to the invention, then these microreactors, preferably these SiC-microreactors, can be used in parallel and/or subsequent arrangements. For example, two, three, four, or more microreactors, preferably two, three, four, or more SiC-microreactors, can be used in parallel and/or subsequent arrangements.

For laboratory search, e.g. on applicable reaction and/or upscaling conditions, without being limited to, for example, as a microreactor the reactor type Plantrix of the company Chemtrix is suitable. Sometimes, if gaskets of a microreactor are made out of other material than HDPTFE, leakage might occur quite soon after short time of operation because of some swelling, so HDPTFE gaskets secure long operating time of microreactor and involved other equipment parts like settler and distillation columns.

For example, an industrial flow reactor (“IFR”, e.g. Plantrix® MR555) comprises of SiC modules (e.g. 3M® SiC) housed within a (non-wetted) stainless steel frame, through which connection of feed lines and service media are made using standard Swagelok fittings. The process fluids are heated or cooled within the modules using integrated heat exchangers, when used in conjunction with a service medium (thermal fluid or steam), and reacted in zig-zag or double zig-zag, meso-channel structures that are designed to give plug flow and have a high heat exchange capacity. A basic IFR (e.g. Plantrix® MR555) system comprises of one SiC module (e.g. 3M® SiC), a mixer (“MRX”) that affords access to A+B→P type reactions. Increasing the number of modules leads to increased reaction times and/or system productivity. The addition of a quench Q/C module extends reaction types to A+B→P1+Q (or C)→P and a blanking plate gives two temperature zones. Herein the terms “A”, “B” and “C” represent educts, “P” and “P1” products, and “Q” quencher. FFKM (perfluororubbers or perfluoroelastomeric compounds containing higher amount of fluorine) is another suitable quite new material applicable for such gaskets (commercially available; also sometimes named FFPM). FFKMs (equivalent to FFPMs) are perfluoroelastomeric compounds containing an even higher amount of fluorine than FKM fluoroelastomers. FKM is a family of fluoroelastomer materials defined by the international standards. It is equivalent to FPM. All FKMs contain vinylidene fluoride as a monomer. They provide additional heat and chemical resistance.

Typical dimensions of an industrial flow reactor (“IFR”, e.g. Plantrix® MR555) are, for example: channel dimensions in (mm) of 4×4 (“MRX”, mixer) and 5×5 (MRH-I/MRH-II; “MRH” denotes residence module); module dimensions (width×height) of 200 mm×555 mm; frame dimensions (width×height) of 322 mm×811 mm. A typical throughput of an industrial flow reactor (“IFR”, e.g. Plantrix® MR555) is, for example, in the range of from about 50 l/h to about 400 l/h. in addition, depending on fluid properties and process conditions used, the throughput of an industrial flow reactor (“IFR”, e.g. Plantrix® MR555), for example, can also be >400 l/h. The residence modules can be placed in series in order to deliver the required reaction volume or productivity. The number of modules that can be placed in series depends on the fluid properties and targeted flow rate.

Typical operating or process conditions of an industrial flow reactor (“IFR”, e.g. Plantrix® MR555) are, for example: temperature range of from about −30° C. to about 200° C.; temperature difference (service−process)<70° C.; reagent feeds of 1 to 3; maximum operating pressure (service fluid) of about 5 bar at a temperature of about 200° C.; maximum operating pressure (process fluid) of about 25 bar at a temperature of about ≤200° C.

### EXAMPLES

The following examples are intended to further illustrate the invention without limiting its scope.

### Example 1

### Batch fluorination of 3-(difluoromethyl)-1-methyl-1H-pyrazole-4-carboxaldehyde (DFMPA) to 3-(difluoromethyl)-5-fluoro-1-methyl-1H-pyrazole-4-carboxylic acid fluoride (5F-DFMPAF)

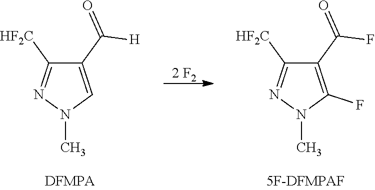

The starting material 3-(difluoromethyl)-1-methyl-1H-pyrazole-4-carboxaldehyde was prepared according to EP2008996 out of difluoroacetone which was prepared out of ethyl difluoroacetoacetate according to EP0623575 and CN103214355.

In a 500 ml PTFE flask with reflux condenser, magnetic stirrer and gas inlet tube in an ice bath, 50 g (0.312 mol) of 3-(difluoromethyl)-1-methyl-1H-pyrazole-4-carboxaldehyde were dissolved in 50 ml anhydrous acetonitrile, a 20% F₂/80% N₂ stream was slowly fed over the inlet pipe into the solution in that manner as the solution did not warm up above 5° C. The conversion of starting material was observed by HPLC. After 1 h of F₂ feed (which was stopped after no exothermic activity could be observed any more), quantitative conversion of the starting material could be detected. Work up. The equimolar formed HF was blown out now using a N₂-stream fed over the inlet tube instead of the F₂-gas. The leaving gas stream was fed into an efficient KOH-scrubber. After no HF could be detected any more in the gas stream leaving the flask, the solvent in the remaining partially crystalline solution was evaporated in vacuum leaving yellow oil. The GC using styrene as internal standard indicated the presence of a 43,4 g 3-(difluoromethyl)-5-fluoro-1-methyl-1H-pyrazole-4-carboxylic acid fluoride (5F-DFMPAF) which corresponds to 71% yield (compounds were analyzed using a Hewlett Packard 6890 Gas Chromatograph 5973 Mass Spectrometer, 50 m Carbowax CP-Sil 8 column).

GC-MS: m/z=196.

### Example 2

### Batch fluorination of 3-(difluoromethyl)-1-methyl-1H-pyrazole-4-carboxylic acid ethylester (DFMP) to 3-(difluoromethyl)-5-fluoro-1-methyl-1H-pyrazole-4-carboxylic acid ethylester (5F-DFMP)

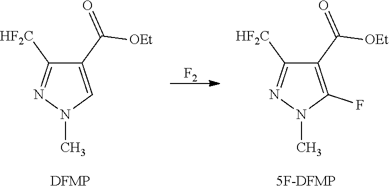

The apparatus used in example 1 on a magnetic stirrer was pre-charged with 50 ml dry CH₃CN. Afterwards 50 g (245 mmol) 3-(difluoromethyl)-1-methyl-1H-pyrazole-4-carboxylic acid ethylester (reference sample bought from Sigma-Aldrich/Merck) prepared according to WO2012/010692 was dissolved and the flask was put into an ice bath. Then 20% F₂-gas was fed over the deep pipe in that manner that the temperature did not exceed 40° C. and until no exothermic activity could be observed any more (˜80 min). Quantitative conversion of the starting material was detected by HPLC. Work up: The equimolar formed HF was blown out now using a N₂-stream fed now over the inlet tube instead of the F₂-gas. The leaving gas stream was collected into an efficient KOH-scrubber. After no HF could be detected any more in the gas stream leaving the flask, the solvent in the remaining oily solution was evaporated in vacuum leaving an oil. GC analysis using styrene as internal standard indicated the presence of 36,5 g 3-(difluoromethyl)-5-fluoro-1-methyl-1H-pyrazole-4-carboxylic acid ethylester which corresponds to 67% yield. Recrystallization from isopropanol/water gave 28 g 3-(difluoromethyl)-5-fluoro-1-methyl-1H-pyrazole-4-carboxylic acid ethylester with 99.2% (GC-)purity.

### Example 3

### Batch fluorination of 3-(chlorodifluoromethyl)-1-methyl-1H-pyrazole-4-carboxylic acid ethylester (CDFMP) to 3-(chlorodifluoromethyl)-5-fluoro-1-methyl-1H-pyrazole-4-carboxylic acid ethylester (5F-CDFMP)

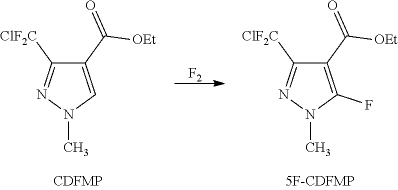

The apparatus used in example 1 on a magnetic stirrer was pre-charged with 50 ml dry CH₃CN. Afterwards 35 g (147 mmol) 3-(chlorodifluoromethyl)-1-methyl-1H-pyrazole-4-carboxylic acid ethylester (CDFMP) prepared according to WO2012010692 was dissolved and the flask was put into an ice bath. Then 20% F₂-gas was fed over the deep pipe in that manner that the temperature did not exceed 50° C. and until no exothermic activity could be observed any more (˜45 min). Quantitative conversion of the starting material was detected by HPLC. Work up: The equimolar formed HF was blown out now using a N₂-stream fed now over the inlet tube instead of the F₂-gas. The leaving gas stream was collected into an efficient KOH-scrubber. After no HF could be detected any more in the gas stream leaving the flask, the solvent in the remaining oily solution was evaporated in vacuum leaving an partially crystallizing oil. Recrystallization from isopropanol/water gave 21.9 g 3-(chlorodifluoromethyl)-5-fluoro-1-methyl-1H-pyrazole-4-carboxylic acid ethylester (5F-CDFMP), a reference for analytics was prepared according to WO 2015/110493. This corresponds to 58% isolated yield. A GC analysis indicated >99.0% purity.

### Example 4

### Batch fluorination of 3-(difluoromethyl)-1-methyl-1H-pyrazole-4-carboxylic acid chloride (DFMAC) to 3-(difluoromethyl)-5-fluoro-1-methyl-1H-pyrazole-4-carboxylic acid fluoride (5F-DFMAF) in a countercurrent apparatus

See FIG. 1 for apparatus and reaction.

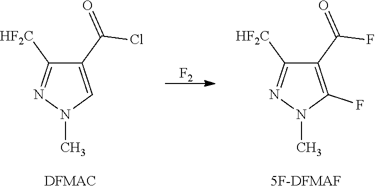

Apparatus: Counter current system was made out of Hastelloy C4 and as drawn and as shown also in CN 111349018. The reservoir is containing the liquid, e.g., oily, starting material or optionally the starting material in an inert solvent.

The pressure during the fluorination is kept by a pressure valve. The inert gas together with (only) very little traces of HF leave as a purge gas during reaction (as HF continuously reacts to the acid fluoride). For the cooler a water cooling system with a water temperature of 8° C. was used. In the countercurrent apparatus the pressure valve at the top was set to 3 bar abs. and had a total volume of 5 l (see drawing), 2.5 l of absolute acetonitrile as solvent together with 200 g (1.03 mol) of 3-(difluoromethyl)-1-methyl-1H-pyrazole-4-carboxylic acid chloride (DFMAC) prepared with SOCl₂ according to example 6 in WO2008/053043 were filled in. The pump was started. When the temperature of the acetonitrile reached 20° C. (coming from 26° C. after start of the loop), the 20% F₂ dosage valve (mass flow meter from Bronkhorst) was opened with a dosage of 1 mol F₂-gas (20% in N₂)/h. All purge gas (N₂) together with most of the formed HF leaves the apparatus over the pressure valve into a basic scrubber system (made out of plastics) together with very little traces of F₂ only. In total, 41.8 g (1.1 mol) F₂ gas (20%) was fed over 1.25 h into that looping reaction mixture, after 1 h feeding the previously observed exothermic activity started to slow down already. Reaction samples were taken very carefully with a stainless steel cylinder (a completely sealed sampling system) every 15 minutes, HF (and volatiles) degassed by evaporation at a vacuum pump and the residue and progress of the reaction was analyzed by HPLC. After stopping dosage, the analysis showed that starting material had disappeared completely (conversion 100% !). Solvent was evaporated in vacuum leaving yellow oil which was confirmed as product by GC-MS with 96% purity which now in such form could be used for many reactions without further purification.

GC-MS: m/z=196.

### Example 5

### 3-(Difluoromethyl)-1-methyl-1H-pyrazole-4-carboxylic acid chloride (DFMAC) to 3-(difluoromethyl)-5-fluoro-1-methyl-1H-pyrazole-4-carboxylic acid fluoride (5F-DFMAF) in a microflow system

See FIG. 2 for apparatus and reaction. (Scheme Microreactor Fluorination)

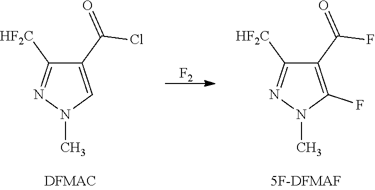

1000 g (5.14 mol) of 3-(difluoromethyl)-1-methyl-1H-pyrazole-4-carboxylic acid chloride were dissolved in 1.0 l dry acetonitrile and put into the starting material reservoir as drawn. The microreactor is a 27 ml volume SiC Microreactor from Chemtrix. The used pump was from company Lewa, each feed was controlled by a Bronkhorst Mass Flow controller.

Highly concentrated F₂ out of a fluorine cell was mixed with 20% N₂ and fed after the pump in stoichiometric amount into the microreactor which is kept at 40° C. by cooling to remove the strong exothermic activity. The feed out of the starting material reservoir was started with 1 l/h, the F₂-feed in stoichiometric amount vs. DFMAC was started right after. The pressure on the system was kept at 3 bar abs. by a pressure valve on the starting material/raw product reservoir (volume 5 l, made out of Hastelloy C4) which was kept at room temperature and which was allowing the inert gas stream content (N₂) together with some HF to leave the apparatus but collects all the raw product as solution in CH₃CN (acetonitrile). HPLC analysis taken in between showed quantitative conversion of starting material. After finishing the feed and closing all valves, the content of the raw product reservoir was degassed by adding slight vacuum (all traces HF also removed) followed by concentration at 20 mbar at a Rotavapor which lead to the product as an yellow oil with 98% purity (confirmed by GC and GC-MS). Remark: It was estimated that this set up of equipment might allow a much higher feed (higher productivity).

### Example 6

Example 5 was repeated but the microreactor was exchanged by a coil reactor made out of Hastelloy C4 (1 m length, diameter: 0.5 cm).

See FIG. 3 for apparatus and reaction.

Highly concentrated F₂ out of a fluorine cell was mixed with 20% N₂ and fed in stoichiometric amounts after the pump into the coil reactor which is kept by external cooling at 40° C. The feed out of the starting material reservoir was started with 1 l/h, and the F₂-feed was started right after. The pressure on the system was also kept at 3 bar abs. by a pressure valve on the starting material/raw product reservoir (volume 5 l, made out of Hastelloy C4) kept at room temperature by cooling but allowing the inert gas stream content (N₂) together with very little traces of HF (˜<1000 ppm) to leave the apparatus but finally all the raw product all over the time was collected there. HPLC analysis taken in between and after 1 h feed showed also a quantitative conversion vs. example 5 but achieved selectivity to 3-(difluoromethyl)-5-fluoro-1-methyl-1H-pyrazole-4-carboxylic acid fluoride was only 77%+mix of by-products. This indicated that in a coil reactor the heat exchange might not have been good enough to avoid hot spots. This trial was not further worked up.

### Example 7

Continuous fluorination of 3-(difluoromethyl)-1-methyl-1H-pyrazole-4-carboxylic acid ethylester (DFMP) to 3-(difluoromethyl)-5-fluoro-1-methyl-1H-pyrazole-4-carboxylic acid ethylester in HF and as solvent with diluted F₂ in a coil reactor made out of Hastelloy C4 (1 m length, diameter: 0.5 cm).

1000 g (4.90 mol) of 3-(difluoromethyl)-1-methyl-1H-pyrazole-4-carboxylic acid ethylester (DFMP) were dissolved (pre-mixed) in 909 g (1.0 l) anhydrous HF and put into the starting material reservoir in the apparatus as drawn and used in example 6. The pump was from Lewa, each feed was controlled by a Bronkhorst Mass Flow controller. F₂ (20% in N₂) out of a cylinder was used as feed in stoichiometric amounts vs. DFMP into the coil reactor which is kept by external cooling during reaction at 35° C. The feed out of the starting material reservoir was started with 1 l/h first, the F₂-feed was started right after. The pressure on the system was also kept at 10 bar abs. by a pressure valve on the starting material/raw product reservoir (volume 5 l, made out of Hastelloy C4), this raw product material trap was kept at room temperature by external cooling but allowing the inert gas stream content (N₂) together with little amounts of HF to leave into an efficient scrubber. HPLC analysis of hydrolyzed and neutralized samples taken 10 min after start of the feed, after 40 minutes and after finish of the feed of the 1000 g DFMP showed with 67% conversion and a selectivity to 3-(difluoromethyl)-5-fluoro-1-methyl-1H-pyrazole-4-carboxylic acid ethylester of 98%+only traces of by-products all similar compositions. This trial was not further worked up.

1. A process for manufacture of 5-fluoro-difluoromethyl-pyrazole compound having the formula (I), 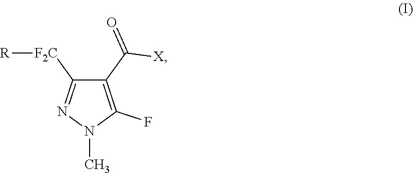
wherein
R represents H (hydrogen atom), Cl (chlorine atom), Br (bromine atom) or F (fluorine atom), and
X represents F (fluorine atom), or a —O—R¹ group wherein R¹ represents a C1-C4-alkyl group, a benzyl group or a substituted benzyl group;
wherein in the process as a starting material a difluoromethyl-pyrazole compound of formula (II), 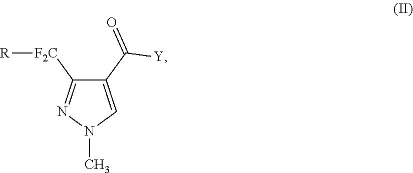
wherein
R represents H (hydrogen atom), Cl (chlorine atom), Br (bromine atom), F (fluorine atom), and
Y represents H (hydrogen atom), Cl (chlorine atom), or a —O—R¹ group wherein R¹ represents a C1-C4-alkyl group, a benzyl group or a substituted benzyl group;
dissolved in an inert solvent [inert solvent=inert organic solvent and/hydrogen fluoride (HF)] is subjected to a direct fluorination reaction with a fluorination gas comprising or consisting of elemental fluorine (F₂), in a reactor which is resistant to elemental fluorine (F₂) and hydrogen fluoride,
to form a reaction mixture containing the 5-fluoro-difluoromethyl-pyrazole compound having the formula (I),
wherein R has the meaning as defined here above and with the provisos that
(i) X in formula (I) is F (fluorine atom) if Y formula (II) is H (hydrogen atom) or Cl (chlorine atom) or Br (bromine atom), and
(ii) X in formula (I) is a —O—R¹ group if Y formula (II) is a —O—R¹ group, and wherein R¹ is as defined here above for X and Y, and wherein R¹ in X has the same meaning as in Y;
and optionally isolating from the reaction mixture and/or purifying, to yield the isolated and/or purified 5-fluoro-difluoromethyl-pyrazole compound having the formula (I).

2. The process according to claim 1, for manufacture of 5-fluoro-difluoromethyl-pyrazole compound having the formula (I), wherein
R represents H (hydrogen atom), Cl (chlorine atom) or F (fluorine atom), and
X represents F (fluorine atom);
wherein in the process as a starting material a difluoromethyl-pyrazole compound of formula (II), wherein
R represents H (hydrogen atom), Cl (chlorine atom) or F (fluorine atom), and
Y represents H (hydrogen atom), Cl (chlorine atom);
dissolved in an inert solvent is subjected to a direct fluorination reaction with a fluorination gas comprising or consisting of elemental fluorine (F₂), in a reactor which is resistant to elemental fluorine (F₂) and hydrogen fluoride,
to form a reaction mixture containing the 5-fluoro-difluoromethyl-pyrazole compound having the formula (I), and optionally isolating from the reaction mixture and/or purifying, to yield the isolated and/or purified 5-fluoro-difluoromethyl-pyrazole compound having the formula (I).

3. The process according to claim 1, for manufacture of 5-fluoro-difluoromethyl-pyrazole compound having the formula (I), wherein
R represents H (hydrogen atom), Cl (chlorine atom) or F (fluorine atom), and
X represents a —O—R¹ group wherein R¹ represents a C1-C4-alkyl group, a benzyl group or a substituted benzyl group, and preferably wherein R¹ represents a C1-C4-alkyl group;
wherein in the process as a starting material a difluoromethyl-pyrazole compound of formula (II), wherein
R represents H (hydrogen atom), Cl (chlorine atom) or F (fluorine atom), and
Y represents a —O—R¹ group wherein R¹ represents a C1-C4-alkyl group, a benzyl group or a substituted benzyl group, and preferably wherein R¹ represents a C1-C4-alkyl group;
dissolved in an inert solvent is subjected to a direct fluorination reaction with a fluorination gas comprising or consisting of elemental fluorine (F₂), in a reactor which is resistant to elemental fluorine (F₂) and hydrogen fluoride,
to form a reaction mixture containing the 5-fluoro-difluoromethyl-pyrazole compound having the formula (I), and optionally isolating from the reaction mixture and/or purifying, to yield the isolated and/or purified 5-fluoro-difluoromethyl-pyrazole compound having the formula (I).

4. A process for manufacture of 5-fluoro-3-(difluoromethyl)-5-fluoro-1-methyl-1H-pyrazole-4-carboxylic acid having the above formula (Ia), 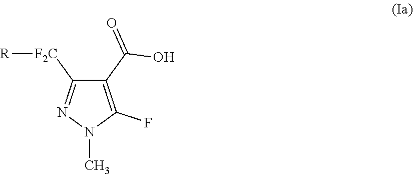
wherein
R represents H (hydrogen atom), Cl (chlorine atom), Br (bromine atom), F (fluorine atom),
and wherein in the process as a starting material a 5-fluoro-difluoromethyl-pyrazole compound having the formula (I), 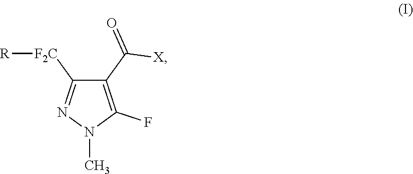
wherein
R represents H (hydrogen atom), Cl (chlorine atom), Br (bromine atom) or F (fluorine atom), and
X represents F (fluorine atom), or a —O—R¹ group wherein R¹ represents a C1-C4-alkyl group, a benzyl group or a substituted benzyl group.
(i) is subjected to a hydrolysis and/or saponification reaction, or
(ii) in case that in the —O—R¹ group the substituent R¹ represents a benzyl group or a substituted benzyl group, is subjected to a hydrolysis and/or saponification reaction, or alternatively to a (mild) catalytic hydrogenation,
to convert the substituent X into a —OH group, and to yield the acid compound 5-fluoro-3-(difluoromethyl)-5-fluoro-1-methyl-1H-pyrazole-4-carboxylic acid having the formula (Ia),
and optionally isolating and/or purifying, to yield the isolated and/or purified acid compound 5-fluoro-3-(difluoromethyl)-5-fluoro-1-methyl-1H-pyrazole-4-carboxylic acid compound having the formula (Ia).

5. The process according to of claim 1, wherein the direct fluorination reaction is carried out until no exothermic activity is observed.

6. The process according to claim 1, wherein the direct fluorination reaction is carried out until no exothermic activity is observed in the reaction mixture.

7. The process according to claim 1, wherein the direct fluorination reaction is carried out at a temperature which does not exceed a temperature of about 55° C., preferably does not exceed a temperature of about 50° C., more preferably does not exceed a temperature of about 45° C., even more preferably does not exceed a temperature of about 40° C., in the reaction mixture.

8. The process according to claim 1, wherein the process is carried out such that HF (hydrogen fluoride) formed in the direct fluorination reaction is eliminated from the reaction mixture by purging the reaction mixture with an inert gas stream until no HF (hydrogen fluoride) is detected in the inert gas stream after it has passed through the reaction mixture.

9. The process according to claim 1, wherein for isolating from the reaction mixture and/or purifying, the reaction mixture is subjected to one or more recrystallization, thereby to yield the isolated and/or purified 5-fluoro-difluoromethyl-pyrazole compound having the formula (I).

10. The process according to any of claim 1, wherein for isolating from the reaction mixture and/or purifying, the reaction mixture is subjected to evaporating the inert solvent under vacuum from the reaction mixture, thereby to obtain as evaporation residue the isolated 5-fluoro-difluoromethyl-pyrazole compound having the formula (I), and optionally further purifying of the evaporation residue to yield the isolated and purified 5-fluoro-difluoromethyl-pyrazole compound having the formula (I).

11. The process according to claim 10, wherein the further purifying of the evaporation residue comprises one or more recrystallization, thereby to yield the isolated and/or purified 5-fluoro-difluoromethyl-pyrazole compound having the formula (I).

12. The process according to claim 1, wherein the elemental fluorine (F₂) is present in the fluorination gas of b) in a (“lower”) concentration in the range of up to about 20% by volume (vol.-%), or approximately about 20% by volume (vol.-%), each based on the total volume of the fluorination gas as 100% by volume.

13. The process according to claim 1, wherein the elemental fluorine (F₂) is present in the fluorination gas of b) in a concentration in the “lower” range of from 0.1% by volume (vol.-%) up to about 20% by volume (vol.-%), in the range of from 0.5% by volume (vol.-%) up to about 20% by volume (vol.-%), in the range of from 1% by volume (vol.-%) up to about 20% by volume (vol.-%), in the range of from 5% by volume (vol.-%) up to about 20% by volume (vol.-%), in the range of from 10% by volume (vol.-%) up to about 20% by volume (vol.-%), in the range of from 15% by volume (vol.-%) up to about 20% by volume (vol.-%), or in a concentration of approximately about 20% by volume (vol.-%), each based on the total volume of the fluorination gas as 100% by volume.

14. The process according to claim 1, wherein the elemental fluorine (F₂) is present in the fluorination gas of b) in a high concentration of at least about 15% by volume, in particular in a high concentration of at least about 20% by volume, preferably in a high concentration of at least about 25% by volume, further preferably of at least about 30% by volume, more preferably of at least about 35% by volume, even more preferably of at least about 45% by volume, each based on the total volume of the fluorination gas as 100% by volume.

15. The process according to claim 1, wherein the fluorine (F₂) is present in the fluorination gas of b) in a high concentration within a range of from about 15-100% by volume, preferably within a range of from about 20-100% by volume, more preferably within a range of from about 25-100% by volume, still more preferably within a range of from about 30-100% by volume, even more preferably within a range of from about 35-100% by volume, an still more preferred within a range of from about 45-100% by volume, each based on the total volume of the fluorination gas as 100% by volume.

16. The process according to any of claim 1, wherein the direct fluorination reaction is carried out in a (closed) column reactor, optionally either operated in a batch manner or operated in a continuous manner, wherein a solution of the starting material difluoromethyl-pyrazole compound dissolved in an inert solvent is circulated in a loop, while the fluorination gas comprising or consisting of elemental fluorine (F₂) in a high concentration is fed into the column reactor and is passed through the liquid medium to react with the starting compound to form a reaction mixture containing the 5-fluoro-difluoromethyl-pyrazole compound having the formula (I), and further circulating in a loop until the fluorination reaction is completed; preferably wherein the loop is operated with a circulation velocity of from 1,500 l/h to 5,000 l/h, more preferably of from 3,500 l/h to 4,500 l/h.

17. The process according to claim 16, wherein the column reactor is equipped with at least one of the following:
(i) at least one cooler (system), at least one liquid reservoir, with inlet and outlet for, and containing as a liquid medium the starting material difluoromethyl-pyrazole compound dissolved in an inert solvent, and as the direct fluorination reaction proceeds also the reaction mixture containing the 5-fluoro-difluoromethyl-pyrazole compound having the formula (I);
(ii) a pump for pumping and circulating the liquid medium of (i) in the column reactor;
(iii) one or more (nozzle) jets, preferably wherein the one or more (nozzle) jets are placed at the top of the column reactor, for spraying the circulating liquid medium of (i) into the column reactor; or alternatively a perforated metal sheet placed at the top of the column reactor, for circulating the liquid medium of (i) into the column reactor, used together with a high-efficiency pump;
(iv) one or more feeding inlets for introducing the fluorination gas comprising or consisting of elemental fluorine (F₂) in a high concentration into the column reactor;
(v) optionally one or more sieves, preferably two sieves, preferably the one or more sieves placed at the bottom of the column reactor;
(vi) and at least one gas outlet equipped with a pressure valve, and at least one outlet for withdrawing the reaction mixture containing the 5-fluoro-difluoromethyl-pyrazole compound having the formula (I).

18. The process according to claim 16, wherein column reactor is a packed bed tower reactor, preferably a packed bed tower reactor which is packed with fillers resistant to elemental fluorine (F₂) and hydrogen fluoride (HF), e.g. with Raschig fillers and/or metal fillers, more preferably wherein the packed bed tower reactor is a gas loop (scrubber) system (tower) which is packed with fillers resistant to elemental fluorine (F₂) and hydrogen fluoride (HF), e.g. Raschig fillers and/or metal fillers.

19. The process according to claim 16, wherein the direct fluorination reaction is carried out with a counter-current flow of the circulating liquid medium of a) comprising or consisting of the starting compound and of the fluorination gas of b) fed into the column reactor and which fluorination gas of b) is comprising or consisting of elemental fluorine (F₂) in a high concentration.

20. The process according to claim 16, wherein the direct fluorination reaction is carried out in a (closed) column reactor, operated in a continuous manner.

21. The process according to claim 16, wherein the direct fluorination reaction is carried out in a (closed) column reactor, which is made out of Hastelloy, preferably which is made out of Hastelloy C4.

22. The process according to claim 1, wherein the direct fluorination reaction is carried out in a coil reactor, operated in a continuous manner.

23. The process according to claim 22, wherein the direct fluorination reaction is carried out in a coil reactor, which is made out of Hastelloy, preferably which is made out of Hastelloy C4.

24. The process according to claim 1, wherein the direct fluorination reaction is carried out in a continuous flow reactor with upper lateral dimensions of about ≤5 mm, or of about ≤4 mm, operated in a continuous manner.

25. The process according to claim 1, wherein the direct fluorination reaction is carried out in a microreactor, operated in a continuous manner.

26. The process according to claim 1, wherein the direct fluorination reaction is carried out in as a continuous process in a microreactor under one or more of the following conditions:
flow rate: of from about 10 ml/h up to about 400 l/h;
temperature: of from about −20° C. up to about 150° C.;
pressure: of from about 1 bar (e.g. 1 atm abs.) up to about 50 bar;
residence time: of from about 1 second, preferably from about 1 minute, up to about 60 minutes.

27. The process according to claim 25, wherein the microreactor is a SiC-microreactor.
